(19) United States
(12) Patent Application Publication
(10) Pub. No.: US 2026/0292316 A1
(43) Pub. Date: Sep. 24, 2026

(54) SYSTEMS AND METHODS FOR PATTERNED ILLUMINATION AND IMAGING

(71) Applicant: President and Fellows of Harvard College, Cambridge, MA (US)

(72) Inventors: Adam Ezra Cohen, Cambridge, MA (US); Yitong Qi, Cambridge, MA (US); Vicente Jose Parot, Cambridge, MA (US)

(73) Assignee: President and Fellows of Harvard College, Cambridge, MA (US)

(21) Appl. No.: 19/476,137

(22) PCT Filed: Apr. 17, 2024
(86) PCT No.: PCT/US2024/024970

§ 371 Date: Oct. 16, 2025

Related U.S. Application Data
(60) Provisional application No. 63/460,250, filed on Apr. 18, 2023.

(51) Int. Cl. H04N 23/10; G02B 21/08; G02B 21/16; G06K 7/14; G06K 19/06; H04N 23/56; H04N 23/73

(52) U.S. Cl. CPC H04N 23/10; G02B 21/08; G02B 21/16; G06K 7/1417; G06K 19/06037; H04N 23/56; H04N 23/73

(57) ABSTRACT

Aspects of the present disclosure relate to systems and methods for combined optical stimulation and fluorescence imaging of a sample (e.g., a biological sample, such as in vivo animal tissue). Certain aspects relate to light source modules configured to emit light at a relatively high intensity (e.g., at least 2 W/cm2 at the sample plane). Certain aspects relate to imaging systems that have a relatively high numerical aperture (e.g., at least 0.4) and are configured to illuminate and image an area having a relatively large diameter (e.g., at least 2 mm). Certain aspects are directed to systems and methods for projecting two or more spatial patterns of illumination at two or more wavelengths on a sample concurrently (e.g., within a single frame of an imaging device, such as a camera).

### CROSS-REFERENCE TO RELATED APPLICATIONS

This application claims the benefit under 35 U.S.C. § 119 (e) of U.S. Provisional Application No. 63/460,250, filed Apr. 18, 2023, entitled “SYSTEMS AND METHODS FOR PATTERNED ILLUMINATION AND IMAGING,” which is hereby incorporated by reference in its entirety.

### GOVERNMENT FUNDING

This invention was made with government support under MH117042 and NS126043 awarded by the National Institutes of Health (NIH). The government has certain rights in this invention.

### FIELD

The present disclosure generally relates to microscopy, such as fluorescence microscopy.

### BACKGROUND

There have been substantial advances in both molecular tools for mediating optical perturbations to cellular activity (e.g., channelrhodopsins, optogenetic silencers, light-activated enzymes) and molecular tools for monitoring cellular activity (e.g., fluorescent reporters, such as calcium reporters and voltage reporters). However, conventional imaging systems generally are unable to capture the full potential of these advances (e.g., by using these molecular tools to perform high-speed voltage imaging in intact tissue and in live animals). Accordingly, improved imaging systems and methods are needed.

### SUMMARY

The present disclosure generally relates to microscopy, such as fluorescence microscopy. The subject matter of the present disclosure involves, in some cases, interrelated products, alternative solutions to a particular problem, and/or a plurality of different uses of one or more systems and/or articles.

In one aspect, a light source module is provided. In some embodiments, the light source module comprises a light source configured to emit a beam of light. In some embodiments, the light source module comprises an enclosure. In certain embodiments, the light source is positioned within the enclosure. In certain embodiments, a surface of the enclosure comprises an optical window positioned within a path of the beam of light. In certain embodiments, an interior volume of the enclosure comprises an inert gas. In some embodiments, the light source module comprises a thermoelectric cooler in thermal communication with the enclosure. In some embodiments, the light source module comprises a heat sink in thermal communication with the thermoelectric cooler.

In another aspect, an imaging system is provided. In some embodiments, the imaging system comprises a first light source module configured to emit a first beam of light at a first wavelength. In some embodiments, the imaging system comprises a second light source module configured to emit a second beam of light at a second wavelength. In some embodiments, the imaging system comprises a first optical system configured to project at least a portion of the first beam of light and/or the second beam of light onto a spatial light modulator configured to produce a spatial pattern of illumination. In some embodiments, the imaging system comprises a second optical system configured to project the spatial pattern of illumination onto a sample being imaged. In some embodiments, the imaging system comprises a back-illuminated CMOS camera having an optical acceptance angle of at least 16°, positioned to receive light from the sample being imaged. In some embodiments, the imaging system comprises a controller configured to modulate current through the first light source module and/or the second light source module, control the spatial light modulator to project a sequence of spatial patterns of illumination onto the sample over time, and/or control exposures of the camera.

In yet another aspect, a method of projecting two or more spatial patterns of illumination at two or more wavelengths concurrently is provided. In some embodiments, the method comprises initiating an exposure of an imaging device for an exposure interval, wherein the exposure interval comprises a plurality of sub-intervals. In some embodiments, the method comprises controlling a first light source to emit light at a first wavelength during a first sub-interval of the exposure interval. In some embodiments, the method comprises controlling a spatial light modulator to project a first spatial pattern of light from the first light source onto a sample during the first sub-interval. In some embodiments, the method comprises controlling a second light source to emit light at a second wavelength during a second sub-interval of the exposure interval. In some embodiments, the method comprises controlling the spatial light modulator to project a second spatial pattern of light from the second light source onto the sample during the second sub-interval. In some embodiments, the method comprises controlling the first light source and the second light source such that the sample is not illuminated during a third sub-interval between the first sub-interval and the second sub-interval.

In still another aspect, a method of imaging a sample is provided. In some embodiments, the method comprises illuminating a biological sample with a first sequence of spatial patterns using light of a first wavelength, wherein the biological sample comprises a plurality of cells expressing one or more fluorescent reporters and one or more light-activated molecules. In some embodiments, the method comprises capturing image data based on light of a second wavelength emitted by the sample using an imaging device. In some embodiments, the method comprises constructing a second spatial pattern configured to provide targeted illumination of cells of the plurality of cells expressing the one or more fluorescent reporters. In some embodiments, the method comprises constructing a third sequence of spatial patterns. In certain embodiments, each spatial pattern of the third sequence of spatial patterns is configured to provide targeted illumination of a subset of cells expressing the one or more fluorescent reporters. In some embodiments, the method comprises illuminating the biological sample with the second spatial pattern at the first wavelength and one or more spatial patterns of the third sequence of spatial patterns at a third wavelength during a stimulus period. In certain embodiments, illumination with the second spatial patterns alternates with illumination with a spatial pattern of the third sequence of spatial patterns at a rate higher than a frame rate of the imaging device.

In still another aspect, a method of registering a light-patterning device to an image capturing device is provided. In some embodiments, the method comprises loading a sample target onto an imaging device; projecting, with the light-patterning device, a pattern of quick response (QR) codes onto the sample target, the pattern of QR codes encoding a plurality of digits corresponding to a number of QR codes in the pattern; acquiring, with the image capturing device, an image of the pattern of QR codes projected onto the sample target; determining a pattern of fiducial marks in the pattern of QR codes based on the acquired image of the pattern of QR codes; and calculating, using the determined pattern of fiducial marks in the pattern of QR codes, a transformation from pixel coordinates of the light-patterning device to pixel coordinates of the image capturing device to obtain a transformed pattern of QR codes. In some embodiments, the method further comprises projecting the transformed pattern of QR codes onto the exposure target and confirming accuracy of the transformed pattern of QR codes based on the projecting the transformed pattern. In some embodiments, the image capturing device comprises a camera. In some embodiments, the imaging device comprises a microscope. In some embodiments, the light-patterning device comprises a digital micromirror device. In some embodiments, the light-patterning device comprises a liquid crystal spatial light modulator.

Other advantages and novel features will become apparent from the following detailed description of various non-limiting embodiments of the invention when considered in conjunction with the accompanying figures.

### BRIEF DESCRIPTION OF THE DRAWINGS

Non-limiting embodiments will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the invention.

In the figures:

FIG. 1 shows a schematic illustration of an exemplary light source module, according to some embodiments.

FIG. 2A shows, according to some embodiments, a schematic illustration of a first view of an exemplary imaging system.

FIG. 2B shows, according to some embodiments, a schematic illustration of a second view of the exemplary imaging system of FIG. 2A.

FIG. 3A shows plots of blue illumination, orange illumination, digital micromirror device (DMD) trigger, camera trigger, DMD pattern, and camera exposure as a function of time for concurrent illumination with blue and orange patterns, according to some embodiments.

FIG. 3B shows plots of blue illumination, orange illumination, DMD trigger, camera trigger, DMD pattern, and camera exposure as a function of time for concurrent illumination with two blue patterns at different intensities and one orange pattern, according to some embodiments.

FIG. 3C shows plots of green illumination, blue illumination, orange illumination, DMD trigger, camera trigger, DMD pattern, and camera exposure as a function of time for concurrent illumination with green, blue, and orange patterns, according to some embodiments.

FIG. 4A shows, according to some embodiments, illumination patterns for spatially registering a DMD and a camera using an affine transformation between DMD and camera pixel coordinates.

FIG. 4B shows, according to some embodiments, illumination patterns for spatially registering a DMD and a camera using a nonlinear transformation to correct for optical distortions.

FIG. 5 shows a process for targeted-illumination voltage imaging in a mouse cortex, according to some embodiments. FIG. 5 shows a wide-field epifluorescence image (upper left), an image of a Hadamard microscopy optical section of the tissue (lower left), locations of identified neurons (lower right), and an image of the sample with illumination patterned to only illuminate the identified neurons (upper right).

FIG. 6A shows, according to some embodiments, a sequence of blue light spatial patterns (e.g., Pattern 1, Pattern 2, . . . , Pattern N) configured to target different subsets of cells.

FIG. 6B shows, according to some embodiments, plots of blue illumination and orange illumination as a function of time. During stimulus periods, blue stimuli (targeted to specific cells) and orange illumination (targeted to all cells) are interleaved. During rest periods, the blue stimulus is turned off and the orange illumination is maintained. Each stimulus pattern (e.g., Pattern 1, Pattern 2, . . . , Pattern N) is repeated m times for later construction of a stimulus-triggered average response in each putative postsynaptic cell. The blue stimulus pattern is then changed and the protocol is repeated until all cells have been stimulated.

FIG. 6C shows, according to some embodiments, the cells (circled) that were identified for stimulation in Pattern 1.

FIG. 7 shows an exemplary design of filter spectra for a wide-area in vivo voltage imaging system.

FIG. 8 shows a workflow for registering a spatial light-patterning device to a camera.

### DETAILED DESCRIPTION

Aspects of the present disclosure relate to systems and methods for combined optical stimulation and fluorescence imaging of a sample (e.g., a biological sample, such as in vivo animal tissue). Certain aspects relate to light source modules configured to emit light at a relatively high intensity (e.g., at least 2 W/cm² at the sample plane). Certain aspects relate to imaging systems that have a relatively high numerical aperture (e.g., at least 0.4) and are configured to illuminate and image an area having a relatively large diameter (e.g., at least 2 mm). Certain aspects are directed to systems and methods for projecting two or more spatial patterns of illumination at two or more wavelengths on a sample concurrently (e.g., within a single frame of an imaging device, such as a camera).

The combination of optical stimulation and fluorescence imaging is a powerful tool for studying numerous biological processes that are structured in space and/or time (e.g., neural activity, cardiac activity, skeletal muscle activity, embryonic development, wound healing, cancer growth). Unlike conventional molecule-based perturbation tools, optical stimulation uses photons for stimulation. Since photons have the ability to be modulated in space, time, and energy (i.e., wavelength) with high resolution, photons can be used to probe how a perturbation delivered to one part of a sample r₁ at a time t₁ can affect dynamics at other parts of the sample r₂ at later times t₂.

The inventors have recognized that conventional imaging systems are unable to capture the full potential of optical stimulation and fluorescence imaging to investigate biological processes due to the inability of conventional imaging systems to provide sufficiently high-intensity spatially patterned optical illumination and wide-area fluorescence imaging. For example, conventional imaging systems are unable to meet the requirements of high-speed in vivo voltage imaging of cellular signaling, which generally include delivering patterned illumination at a relatively high intensity (e.g., at least 2 W/cm² at the sample plane), illuminating and imaging a relatively large area (e.g., an area having a largest dimension of at least 2 mm), collecting light with a relatively high numerical aperture (e.g., at least 0.4), and imaging at a relatively low magnification (e.g., 3× or less). The systems and methods described herein address the deficiencies of conventional imaging systems and methods. Accordingly, some embodiments are directed to systems and methods for providing high-intensity illumination. Some embodiments are directed to systems and methods for providing wide-area imaging with a relatively high numerical aperture and a relatively low magnification.

The inventors have also recognized that, in order to capture the full potential of optical stimulation and fluorescence imaging to investigate biological processes, it may be desirable to project two or more spatial patterns of illumination at two or more wavelengths (e.g., a first wavelength for stimulating a light-activated molecule, a second wavelength for exciting a fluorescent reporter) concurrently (e.g., within a single frame of an imaging device, such as a camera). Two or more spatial patterns of illumination may be considered to be projected concurrently if they are projected within a single frame of an imaging device (e.g., a camera). In some cases, using a single spatial light modulator (e.g., digital micromirror device (DMD)) to project the two or more spatial patterns of illumination may advantageously reduce costs and/or allow for the projection of complex patterns of illumination.

According to certain embodiments described herein, two or more spatial patterns of illumination are interleaved on a single spatial light modulator while synchronously modulating the illumination wavelengths. If the frequency at which the spatial light modulator cycles between the two or more spatial patterns is faster than the response kinetics of the light-sensitive molecules of the sample being imaged, the light-responsive molecules effectively experience time-average illumination rather than rapid fluctuations. To implement this approach, the images projected by the spatial light modulator should be precisely registered in space and time with the images acquired on the imaging device. Accordingly, certain embodiments are directed to systems and methods for performing spatial and/or temporal registration of spatial light modulator images and imaging device images.

Some aspects are directed to light source modules configured to provide high-intensity illumination. The inventors have recognized that investigation of biological processes using optical stimulation and fluorescence imaging may require illumination at relatively high intensities (e.g., at least 2 W/cm² at the sample plane). However, commercially available light-emitting diodes (LEDs) are configured to produce light at much lower intensities. Accordingly, improved light source modules are needed.

In some embodiments, a light source module is configured to provide a light source emitting light at a relatively high intensity. The inventors have recognized that reducing the temperature of a light source may advantageously increase its intensity. Accordingly, some aspects are directed to a light source module comprising a thermoelectric cooler and/or a heat sink such that a light source is maintained at a relatively low temperature. The inventors have also recognized that increasing the current driving a light source may advantageously increase its intensity. Accordingly, some aspects are directed to a light source module comprising a light source driven at a relatively high current.

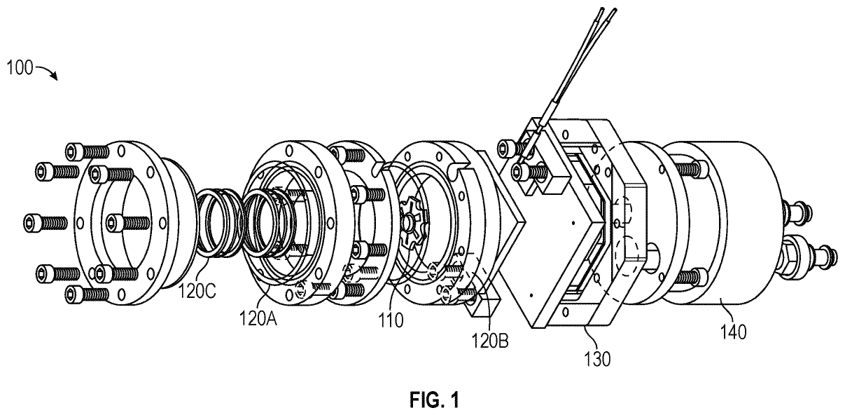

FIG. 1 shows a schematic illustration of an exemplary light source module 100. As shown in FIG. 1, light source module 100 comprises light source 110, enclosure 120, thermoelectric cooler 130, and heat sink 140. In FIG. 1, enclosure 120 comprises a top portion 120A and a bottom portion 120B. Top portion 120A comprises an optical window 120C. Light source 110 is positioned within an interior volume formed by top portion 120A and bottom portion 120B. In certain embodiments, the interior volume may comprise an inert gas. Enclosure 120 is in thermal communication with thermoelectric cooler 130 since bottom portion 120B of enclosure 120 is in direct physical contact with a surface (e.g., a cold surface) of thermoelectric cooler 130. Thermoelectric cooler 130 is in thermal communication with heat sink 140 since a surface (e.g., a hot surface) of thermoelectric cooler 130 is in direct physical contact with heat sink 140.

In operation, light source 110 emits a beam of light, and at least a portion of the beam of light is transmitted through optical window 120C of enclosure 120. Light source 110 also generates heat, at least a portion of which is transferred to thermoelectric cooler 130. At least a portion of the heat transferred to thermoelectric cooler 130 is subsequently transferred from thermoelectric cooler 130 to heat sink 140.

In some embodiments, a light source module comprises a light source positioned within an enclosure (e.g., an inner volume of an enclosure). In some embodiments, the light source comprises a light-emitting diode (LED). In some cases, employing an LED as a light source may result in substantial cost savings compared to using alternative light sources (e.g., lasers). In certain embodiments, the light source consists of a single LED, although more than one LED and/or other light source may be used in other embodiments. In some cases, an application may require focusing light on a relatively small area, and it may therefore be advantageous for the light source to comprise a single LED instead of multiple LEDs. In some embodiments, the light source comprises a laser and/or an incandescent lamp.

In some embodiments, the light source emits light having a relatively high intensity. In certain cases, the light source emits light having an intensity (e.g., at the sample plane) of at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm². In certain embodiments, the light source emits light having an intensity (e.g., at the sample plane) in a range from 2 W/cm² to 5 W/cm², 2 W/cm² to 10 W/cm², 3 W/cm² to 5 W/cm², 3 W/cm² to 10 W/cm², 5 W/cm² to 10 W/cm², or 8 W/cm² to 10 W/cm².

The light source may emit light having any wavelength. In some embodiments, the light source emits light having a peak wavelength in a range from 380 nm to 450 nm, 400 nm to 700 nm, 450 nm to 495 nm, 495 nm to 565 nm, 565 nm to 590 nm, 580 nm to 600 nm, 590 nm to 625 nm, or 625 nm to 750 nm. In certain embodiments, the light source emits light having a peak wavelength of approximately 380 nm, 400 nm, 450 nm, 470 nm, 495 nm, 565 nm, 580 nm, 600 nm, 610 nm, 625 nm, or 750 nm.

In some embodiments, an interior volume of the enclosure is maintained at a relatively low temperature (e.g., such that the light source is at a relatively low temperature) during operation of the light source module. In certain embodiments, the interior volume of the enclosure has a temperature of −30° C. or less, −35° C. or less, or −40° C. or less during operation of the light source module. In certain embodiments, the interior volume of the enclosure has a temperature in a range from −40° C. to −35° C., −40° C. to −30° C., or −35° C. to −30° C. during operation of the light source module. In some cases, a light source operated at a relatively low temperature described herein emits light having an intensity that is at least 1.6, at least 1.7, at least 1.8, at least 1.9, at least 2.0, or at least 2.5 times higher than an intensity of light emitted by the same light source operated at 25° C. In some cases, a light source operated at a relatively low temperature described herein emits light having an intensity that is 1.6 to 2.0, 1.6 to 2.5, 1.8 to 2.5, or 2.0 to 2.5 times higher than an intensity of light emitted by the same light source operated at 25° C.

In some embodiments, the light source is configured to be driven at a relatively high current during operation of the light source module. In certain embodiments, the light source is configured to be driven at a current of at least 1 A, at least 2 A, at least 3 A, at least 4 A, at least 5 A, at least 8 A, or at least 10 A. In certain embodiments, the light source is configured to be driven at a current in a range from 1 A to 3 A, 1 A to 5 A, 1 A to 8 A, 1 A to 10 A, 3 A to 5 A, 3 A to 8 A, 3 A to 10 A, 5 A to 10 A, or 8 A to 10 A. In some cases, a light source operated at a relatively high current described herein emits light having an intensity that is at least 2.0, at least 3.0, at least 4.0, or at least 5.0 times higher than an intensity of light emitted by the same light source operated at a current of 350 mA. In some cases, a light source operated at a relatively high current described herein emits light having an intensity that 2.0 to 5.0 or 3.0 to 5.0 times higher than an intensity of light emitted by the same light source operated at a current of 350 mA.

The enclosure may have any shape. In some embodiments, an inner volume of the enclosure is substantially cylindrical, substantially prismatic, or irregularly shaped. The enclosure may be formed of any suitable material. The material may comprise a polymer (e.g., an acrylic), a ceramic, and/or a metal, etc.

In some embodiments, a surface of the enclosure comprises an optical window positioned within a path of the beam of light emitted by the light source (e.g., to allow transmission of light emitted by the light source). The optical window may have any suitable size and shape. In certain instances, the optical window has a largest dimension (e.g., diameter) of at least 5 mm, at least 10 mm, at least 20 mm, at least 30 mm, at least 40 mm, at least 50 mm, at least 80 mm, or at least 100 mm. In certain instances, the optical window has a largest dimension (e.g., diameter) in a range from 5 mm to 10 mm, 5 mm to 20 mm, 5 mm to 50 mm, 5 mm to 80 mm, 5 mm to 100 mm, 10 mm to 20 mm, 10 mm to 50 mm, 10 mm to 80 mm, 10 mm to 100 mm, 20 mm to 50 mm, 20 mm to 80 mm, 20 mm to 100 mm, or 50 mm to 100 mm. In some instances, the optical window has a diameter of approximately 5 mm, 10 mm, 20 mm, 30 mm, 40 mm, 50 mm, 50.8 mm, 80 mm, or 100 mm.

In some embodiments, an interior volume of the enclosure comprises an inert gas. In some cases, the inert gas may advantageously prevent condensation when the light source is maintained below the dew point. Non-limiting examples of an inert gas include nitrogen, helium, argon, neon, krypton, and xenon. More than one inert gas may be used in some cases.

In some embodiments, the enclosure of a light source module is in thermal communication with a thermoelectric cooler. A thermoelectric cooler generally refers to a cooler in which heat is transferred from a first surface (e.g., a cold surface) to a second surface (e.g., a hot surface) when an electric current runs through the device. A non-limiting example of a suitable thermoelectric cooler is Sheetak SKHC1-199-16-T100-SS-TF00-ALO. As used herein, two or more components are in “thermal communication” if heat transfer can occur between the two or more components due to direct or indirect physical contact of the two or more components. Two or more components that are solely connected by air or by the ground are generally not considered to be in thermal communication. In certain embodiments, the enclosure of the light source module is in direct physical contact with a surface (e.g., a cold surface) of the thermoelectric cooler. In certain embodiments, the enclosure of the light source module is in indirect physical contact with a surface (e.g., a cold surface) of the thermoelectric cooler. In some embodiments in which the enclosure is in indirect physical contact with the thermoelectric cooler, the enclosure and the thermoelectric cooler are separated by one or more intervening components, where each intervening component comprises (or, in some cases, consists of) a material having a relatively high thermal conductivity (e.g., at least 100 W/(m·K)). Non-limiting examples of materials having a relatively high thermal conductivity include a metal (e.g., silver, copper, gold, aluminum, tungsten, zinc), a ceramic (e.g., silicon carbide, aluminum nitride), diamond, graphite, and a thermal adhesive or paste. In certain embodiments, the enclosure is connected to the thermoelectric cooler via a gas-tight connection. In some cases, more than one such material may be used.

In some embodiments, the thermoelectric cooler (e.g., the hot surface of the thermoelectric cooler) is in thermal communication with a heat sink. The heat sink may be any component configured to transfer heat from a higher temperature device (e.g., a light source, a thermoelectric cooler) to a fluid medium (e.g., air, water). In certain embodiments, the heat sink is liquid-cooled heat sink (e.g., a water-cooled heat sink). A non-limiting example of a suitable water-cooled heat sink is EK-Quantum Velocity. In certain embodiments, the heat sink is an air-cooled heat sink. In certain embodiments, a surface (e.g., a hot surface) of the thermoelectric cooler is in direct physical contact with a heat sink. In certain embodiments, a surface (e.g., a hot surface) of the thermoelectric cooler is in indirect physical contact with a heat sink. In some embodiments in which the thermoelectric cooler is in indirect physical contact with the heat sink, the thermoelectric cooler and the heat sink are separated by one or more intervening components, where each intervening component comprises (or, in some cases, consists of) a material having a relatively high thermal conductivity (e.g., at least 100 W/(m·K)). Non-limiting examples of materials having a relatively high thermal conductivity Include a metal (e.g., silver, copper, gold, aluminum, tungsten, zinc), a ceramic (e.g., silicon carbide, aluminum nitride), diamond, graphite, and a thermal adhesive or paste.

In some embodiments, the light source module comprises one or more lenses and/or filters and/or other optical components positioned within the path of a beam of light emitted by the light source of the light source module. In some embodiments, one or more lenses and/or filters are positioned inside the enclosure. In certain embodiments, for example, a collimating lens is positioned between the light source and the optical window of the enclosure. The collimating lens may advantageously reduce or prevent wide angular spread of light emitted by the light source, which could result in the light diverging too much by the time it passed the optical window. In some instances, the collimating lens comprises an aspheric condenser lens, Ø1″, focal length 16 mm, with an anti-reflective coating from 350-700 nm. In certain cases where the light source emits substantially blue light, the light source module may further comprise an achromat. In some instances, the achromat comprises a Ø1″ 10 cm focal length achromat. In certain embodiments, an excitation filter is positioned between the light source and the optical window of the enclosure. In some cases, the excitation filter removes the long-wavelength component of light emitted by the light source to avoid contamination of a fluorescence signal from a fluorescent reporter on the imaging device. A non-limiting example of a suitable excitation filter is a Chroma ET580/25x-25mmR filter.

In some embodiments, one or more lenses and/or filters are positioned outside the enclosure. In certain embodiments, a plano-convex lens is positioned outside the enclosure and within the path of a beam of light emitted by the light source of the light source module. In some cases, the plano-convex lens may further collimate the beam of light. A non-limiting example of a suitable plano-convex lens is a Ø2″, focal length 10 cm (Thorlabs LA1050-A-ML) lens.

Some embodiments are directed to a system comprising two or more light source modules. In certain embodiments, the system comprises a filter. A non-limiting example of a suitable filter is a 60×85 mm long-pass dichroic with a cutoff wavelength of 580 nm. In certain embodiments, the system comprises a lens. In some cases, the lens may focus light from a first light source module and a second light source module onto a digital micromirror device (DMD). In certain cases, the lens may be a plano-convex lens. A non-limiting example of a suitable plano-convex lens is a Thorlabs LA1238-A, Ø75.0 mm, f=100 mm, AR Coating: 350-700 nm lens.

Some aspects are directed to an imaging system. In certain embodiments, the imaging system comprises a spatial pattern projection system and a wide field-of-view (FOV) epifluorescence microscope.

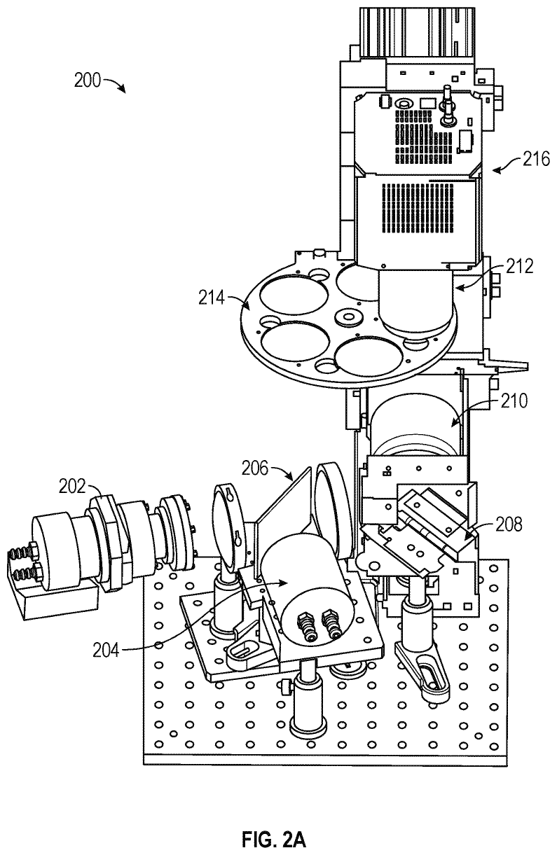

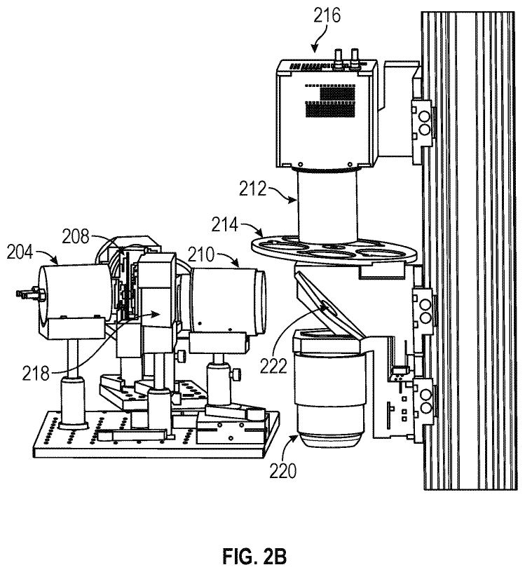

FIG. 2A shows a schematic illustration of a first view of an exemplary imaging system 200. As shown in FIG. 2A, imaging system 200 comprises first light source module 202, second light source module 204, dichroic beam combiner 206, spatial light modulator 208, projection lens 210, tube lens 212, emission filter 214, and imaging device 216. FIG. 2B shows a schematic illustration of a second view of exemplary imaging system 200. In addition to the components shown in FIG. 2A, FIG. 2B shows that imaging system 200 further comprises total internal reflection (TIR) prism 218, objective lens 220, and dichroic mirror 222. In some embodiments, imaging system 200 comprises a controller (not shown in FIG. 2A or 2B) configured to control one or more components of imaging system 200 (e.g., first light source module 202, second light source module 204, spatial light modulator 208, and/or imaging device 216).

In operation, a sample (e.g., a biological sample comprising tissue comprising a light-activated molecule configured to be stimulated at a first wavelength and a fluorescent reporter configured to be excited at a second wavelength) is positioned in a suitable position for imaging (e.g., below objective lens 220). First light source module 202 may emit light having the first wavelength and second light source module 204 may emit light having the second wavelength (or, alternatively, second light source module 204 may emit light having the first wavelength and first light source module 202 may emit light having the second wavelength). Light emitted by first light source 202 and/or light emitted by second light source 204 may be combined by dichroic beam combiner 206 and reflected and/or transmitted onto TIR prism 218. TIR prism 218 may direct light onto spatial light modulator 208. Spatial patterns projected by spatial light modulator 208 may be collected by tube lens 212 and directed into objective lens 220 via dichroic mirror 222. Fluorescent emission from the sample may be collected by objective lens 220, filtered by emission filter 214, and reimaged onto imaging device 216 through tube lens 212. All microscope hardware may be synchronized by a data acquisition device and controlled by software.

In some embodiments, the imaging system comprises a first light source module. The first light source module may be any light source module described herein. In some embodiments, the first light source module comprises a light source (e.g., an LED) configured to emit a beam of light having a first wavelength. In some embodiments, the imaging system comprises a second light source module. The second light source module may be any light source module described herein. In some embodiments, the second light source module comprises a light source (e.g., an LED) configured to emit a beam of light having a second wavelength. In some embodiments, the second wavelength is different from the first wavelength. In some embodiments, the first wavelength and/or the second wavelength are independently in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In some embodiments, the first wavelength and/or the second wavelength are independently approximately 380 nm, 400 nm, 450 nm, 470 nm, 495 nm, 565 nm, 610 nm, 625 nm, or 750 nm. In certain embodiments, the first wavelength is substantially blue (e.g., in a range from about 450 nm to 495 nm) and the second wavelength is substantially orange (e.g., in a range from about 590 nm to 625 nm) (or the first wavelength is substantially orange and the second wavelength is substantially blue). In certain embodiments, the first wavelength is substantially blue (e.g., in a range from about 450 nm to 495 nm) and the second wavelength is substantially red (e.g., in a range from about 625 nm to 750 nm) (or the first wavelength is substantially red and the second wavelength is substantially blue).

In some embodiments, the first light source module and/or the second light source module are configured to emit light at a relatively high intensity. In certain cases, the first light source module and/or the second light source module are configured to emit light having an intensity (e.g., at the sample plane) of at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm². In certain embodiments, the first light source module and/or the second light source module are configured to emit light having an intensity (e.g., at the sample plane) in a range from 2 W/cm² to 5 W/cm², 2 W/cm² to 10 W/cm², 3 W/cm² to 5 W/cm², 3 W/cm² to 10 W/cm², 5 W/cm² to 10 W/cm², or 8 W/cm² to 10 W/cm².

In some embodiments, the imaging system comprises more than two light source modules. In certain embodiments, for example, the imaging system comprises three, four, five, six, seven, or more light source modules.

In some embodiments, the imaging system comprises a first optical system configured to project at least a portion of the first beam of light and/or the second beam of light onto a spatial light modulator. In some embodiments, the spatial light modulator is placed in an illumination path to project one or more spatial patterns of illumination onto a sample being imaged.

In some embodiments, the first optical system comprises a dichroic beam combiner. In certain embodiments, the dichroic beam combiner is configured to reflect and/or transmit the first beam of light. In certain embodiments, the dichroic beam combiner is configured to reflect and/or transmit the second beam of light. In some cases, the dichroic beam combiner functions as a long-pass filter (e.g., with a cutoff wavelength of 580 nm). A non-limiting example of a suitable dichroic beam combiner is a Semrock, Di01-R488/543/635, 60×85 mm dichroic mirror.

In some embodiments, the first optical system comprises one or more lenses and/or prisms configured to focus light emitted from the first light source module and/or the second light source module onto the spatial light modulator. In some embodiments, the first optical system comprises a total internal reflection (TIR) prism. The TIR prism may be configured to direct incident light onto the spatial light modulator. In certain embodiments, the TIR prism comprises two laminated prisms. A non-limiting example of a suitable TIR prism is a Young Optics, Inc., 72.CAC14G001 prism. In some embodiments, the first optical system comprises a plano-convex lens. The plano-convex lens may be configured to focus incident light onto the spatial light modulator. A non-limiting example of a plano-convex lens is a Thorlabs LA1238-A, Ø75.0 mm, f=100 mm, AR Coating: 350-700 nm lens. Other types of lenses and/or prisms may be included in the first optical system.

In some embodiments, the imaging system comprises a spatial light modulator. In certain embodiments, the spatial light modulator comprises a digital micromirror device (DMD). The DMD may comprise an array of tilting micromirrors that can be individually addressed to direct light into or away from an optical path. In general, each micromirror, or pixel, may be in a positive state tilted toward the illumination (referred to as the “on” state) or in a negative state tilted away from the illumination (referred to as the “off” state). Each pixel of the DMD may be imaged onto a corresponding location in a sample plane. By modulating the pattern on the DMD, a binary spatial pattern of illumination may be projected onto a sample. In some cases, the array of tilting micromirrors is a rectangular or other regular array. In some cases, the array of tilting micromirrors comprises hundreds of thousands or millions of mirrors. In certain embodiments, for example, a DMD comprises an array of 1024×768 mirrors, 768×576 mirrors, 2048×1152 mirrors, or 3840× 2160 mirrors. A non-limiting example of a suitable DMD is a Vialux V-7001 DMD comprising 1024×768 pixels with a 13.7 μm pitch.

In some cases, another device able to project spatial patterns of illumination may be used instead of or in addition to a DMD. For example, a liquid crystal and a polarizer may be used to project spatial patterns of illumination. Alternatively, light source module 202 and/or light source module 204 may comprise an array of LEDs that produces spatial patterns without the need for a separate spatial light modulator.

In some embodiments, the imaging system comprises a second optical system configured to project the spatial pattern of illumination onto a sample being imaged. In certain embodiments, the second optical system comprises one or more lenses and/or dichroic mirrors. In certain cases, the one or more lenses comprise a projection lens and/or an objective lens. The magnification of the second optical system may be in a range from 0.5× to 5×.

In some embodiments, the second optical system comprises a projection lens. The projection lens may be configured to collect patterned light from the spatial light modulator. In certain embodiments, the projection lens is a tube lens (e.g., a 100 mm tube lens). A non-limiting example of a suitable projection lens is a Zeiss, Makro-Planar 100 mm lens.

In some embodiments, the second optical system comprises a dichroic mirror. The dichroic mirror may be configured to reflect patterned light from the projection lens into an objective lens. In some embodiments, the dichroic mirror may be configured to transmit fluorescence emission from the sample. A non-limiting example of a suitable dichroic mirror is an Alluxa, 504/605 ULTRA Dualband Dichroic Beamsplitter, 60×85 mm, 5 mm thick substrate mirror.

In some embodiments, the second optical system comprises an objective lens. A non-limiting example of a suitable objective lens is an Olympus, MVPLAPO 2XC, NA 0.5 lens having a focal length of 50 mm.

In some embodiments, the imaging system comprises one or more optical elements configured to collect fluorescent emission from the sample. In some embodiments, for example, fluorescent emission is collected by the objective lens. In certain embodiments, the imaging system further comprises an emission filter positioned within an optical path between the sample and the imaging device. A non-limiting example of an emission filter is a Semrock, FF02-641/75, 60 mm diameter emission filter.

In some embodiments, the fluorescent emission is focused onto an imaging device through a tube lens positioned within an optical path between the sample and the imaging device. In certain embodiments, the tube lens is a 50 mm tube lens (e.g., a 50 mm f/0.85 lens). A non-limiting example of a suitable 50 mm tube lens is a VS Technology, VS-50085/M42 lens.

In some embodiments, the imaging system comprises an imaging device positioned to receive fluorescent emissions from the sample. In certain embodiments, the imaging device is a back-illuminated CMOS camera (e.g., a back-illuminated scientific CMOS camera). In certain embodiments, the back-illuminated CMOS camera has an optical acceptance angle of at least 16°, at least 20°, at least 25°, at least 30°, at least 35°, or at least 40°. In certain embodiments, the back-illuminated CMOS camera has an optical acceptance angle in a range of 16° to 20°, 16° to 25°, 16° to 30°, 16° to 35°, 16° to 40°, 20° to 25°, 20° to 30°, 20° to 35°, 20° to 40°, 25° to 30°, 25° to 35°, 25° to 40°, 30° to 35°, 30° to 40°, or 35° to 40°. In some cases, the back-illuminated CMOS camera does not comprise an internal microlens array and therefore can accept incident light at high angles. In some embodiments, the imaging device has a frame rate of at least 400 Hz, at least 450 Hz, at least 500 Hz, at least 800 Hz, at least 1 kHz, or at least 1.5 kHz during operation of the imaging system. In certain embodiments, the imaging device has a frame rate in a range of 400 Hz to 500 Hz, 400 Hz to 800 Hz, 400 Hz to 1 kHz, 400 Hz to 1.5 kHz, 500 Hz to 800 Hz, 500 Hz to 1 kHz, 500 Hz to 1.5 kHz, 800 Hz to 1 kHz, 800 Hz to 1.5 kHz, or 1 to 1.5 kHz during operation of the imaging system.

In some embodiments, the imaging system comprises a controller configured to modulate current through the first light source module and/or the second light source module, control the spatial light modulator to project a sequence of spatial patterns of illumination onto the sample over time, and/or control exposures of the camera.

In some embodiments, the magnification of the imaging system is 2.5× or less, 2× or less, 1.5× or less, or 1× or less. In certain embodiments, the magnification of the imaging system is in a range from 1× to 1.5×, 1× to 2×, 1× to 2.5×, 1.5× to 2×, 1.5× to 2.5×, or 2× to 2.5×.

In some embodiments, the imaging system is configured to image an area having a largest dimension (e.g., diameter) of at least 2 mm, at least 3 mm, at least 4 mm, at least 5 mm, at least 10 mm, at least 15 mm, or at least 20 mm. In certain embodiments, the imaging system is configured to image an area having a largest dimension (e.g., diameter) in a range from 2 mm to 5 mm, 2 mm to 10 mm, 2 mm to 15 mm, 2 mm to 20 mm, 3 mm to 5 mm, 3 mm to 10 mm, 3 mm to 15 mm, 3 mm to 20 mm, 5 mm to 10 mm, 5 mm to 15 mm, 5 mm to 20 mm, 10 mm to 15 mm, 10 mm to 20 mm, or 15 mm to 20 mm.

Some aspects are directed to methods of projecting two or more spatial patterns of illumination at two or more wavelengths concurrently (e.g., within a single frame of an imaging device). In some cases, such methods may advantageously allow a sample to be illuminated by a first spatial pattern at a first wavelength configured to stimulate one or more light-activated molecules in the sample and a second spatial pattern at a second wavelength configured to excite one or more fluorescent reporters in the sample. In some embodiments, this combines targeted optogenetic stimulation with structured illumination microscopy in order to map functional connections between cells.

In some embodiments, a single spatial light modulator (e.g., DMD) is used to project two or more spatial patterns of illumination at two or more wavelengths concurrently. In some cases, using a single spatial light modulator may advantageously reduce costs compared to using two or more spatial light modulators. In certain instances, however, two or more spatial light modulators may be used to project two or more spatial patterns of illumination at two or more wavelengths concurrently.

In some embodiments, a method of projecting two or more spatial patterns of illumination at two or more wavelengths concurrently comprises initiating an exposure of an imaging device (e.g., a camera) for an exposure interval comprising a plurality of sub-intervals. In certain embodiments, the exposure interval has a duration of at least 50 μs, at least 100 μs, at least 500 μs, at least 800 μs, at least 1 ms, at least 1.25 ms, at least 1.5 ms, at least 2 ms, at least 5 ms, at least 10 ms, at least 50 ms, at least 100 ms, or at least 500 ms. In certain embodiments, the exposure interval has a duration between 50 μs and 100 μs, 50 μs and 500 μs, 50 μs and 800 μs, 50 μs and 1 ms, 50 μs and 1.25 ms, 50 μs and 1.5 ms, 50 μs and 2 ms, 50 μs and 5 ms, 50 μs and 10 ms, 50 μs and 50 ms, 50 μs and 100 ms, 50 μs and 500 ms, 100 μs and 500 μs, 100 μs and 800 μs, 100 μs and 1 ms, 100 μs and 1.25 ms, 100 μs and 1.5 ms, 100 μs and 2 ms, 100 μs and 5 ms, 100 μs and 10 ms, 100 μs and 50 ms, 100 μs and 100 ms, 100 μs and 500 ms, 500 μs and 1 ms, 500 μs and 1.25 ms, 500 μs and 1.5 ms, 500 μs and 2 ms, 500 μs and 5 ms, 500 μs and 10 ms, 500 μs and 50 ms, 500 μs and 100 ms, 500 μs and 500 ms, 1 ms and 1.5 ms, 1 ms and 2 ms, 1 ms and 5 ms, 1 ms and 10 ms, 1 ms and 50 ms, 1 ms and 100 ms, 1 ms and 500 ms, 5 ms and 10 ms, 5 ms and 50 ms, 5 ms and 100 ms, 5 ms and 500 ms, 10 ms and 50 ms, 10 ms and 100 ms, 10 ms and 500 ms, 50 ms and 100 ms, 50 ms and 500 ms, or 100 ms and 500 ms.

In some embodiments, the method of projecting two or more spatial patterns of illumination at two or more wavelengths concurrently comprises controlling a first light source to emit light at a first wavelength during a first sub-interval of the exposure interval. In some embodiments, the method comprises controlling a spatial light modulator (e.g., a DMD) to project a first spatial pattern of light at the first wavelength onto the sample during the first sub-interval. In some embodiments, the first sub-interval has a duration of 1 ms or less, 800 μs or less, 500 μs or less, 250 μs or less, 200 μs or less, 150 μs or less, 100 μs or less, 50 μs or less, or 20 μs or less. In some embodiments, the first sub-interval has a duration in a range from 20 μs to 50 μs, 20 μs to 100 μs, 20 μs to 200 μs, 20 μs to 500 μs, 20 us to 1 ms, 50 μs to 100 μs, 50 μs to 200 μs, 50 μs to 500 μs, 50 μs to 1 ms, 100 μs to 200 μs, 100 μs to 500 μs, 100 μs to 1 ms, 200 μs to 500 μs, 200 μs to 1 ms, or 500 μs to 1 ms. In some embodiments, the first sub-interval has a duration that is about 50% or less, 40% or less, 30% or less, 20% or less, or 10% or less of the duration of the exposure interval. In certain embodiments, the first sub-interval has a duration that is in a range of 10% to 20%, 10% to 30%, 10% to 40%, 10% to 50%, 20% to 40%, 20% to 50%, 30% to 50%, or 40% to 50% of the duration of the exposure interval.

In some embodiments, the first wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In some embodiments, the first wavelength is approximately 380 nm, 400 nm, 450 nm, 470 nm, 495 nm, 565 nm, 610 nm, 625 nm, or 750 nm. In certain embodiments, light at the first wavelength is emitted from the first light source at an intensity (e.g., at the sample plane) of at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm². In certain embodiments, light at the first wavelength is emitted from the first light source at an intensity (e.g., at the sample plane) in a range from 2 W/cm² to 5 W/cm², 2 W/cm² to 10 W/cm², 3 W/cm² to 5 W/cm², 3 W/cm² to 10 W/cm², 5 W/cm² to 10 W/cm², or 8 W/cm² to 10 W/cm².

In some embodiments, the method of projecting two or more spatial patterns of illumination at two or more wavelengths concurrently comprises controlling a second light source to emit light at a second wavelength during a second sub-interval of the exposure interval. In some embodiments, the method comprises controlling the spatial light modulator to project a second spatial pattern of light at the second wavelength onto the sample during the second sub-interval. In some embodiments, the first sub-interval and the second sub-interval do not overlap. The first sub-interval may occur before the second sub-interval or the second sub-interval may occur before the first sub-interval. In some embodiments, the second sub-interval has a duration of 100 ms or less, 50 ms or less, 10 ms or less, 1 ms or less, 800 μs or less, 500 μs or less, 250 μs or less, 200 μs or less, 150 μs or less, 100 μs or less, or 50 μs or less. In some embodiments, the second sub-interval has a duration in a range from 50 μs to 100 μs, 50 μs to 200 μs, 50 μs to 500 μs, 50 μs to 1 ms, 50 μs to 10 ms, 50 μs to 50 ms, 50 μs to 100 ms, 100 μs to 200 μs, 100 μs to 500 μs, 100 μs to 1 ms, 100 μs to 10 ms, 100 μs to 50 ms, 100 μs to 100 ms, 200 μs to 500 μs, 200 μs to 1 ms, 200 μs to 10 ms, 200 μs to 50 ms, 200 μs to 100 ms, 500 μs to 1 ms, 500 μs to 10 ms, 500 μs to 50 ms, 500 μs to 100 ms, 1 ms to 10 ms, 1 ms to 50 ms, 1 ms to 100 ms, 10 ms to 50 ms, 10 ms to 100 ms, or 50 ms to 100 ms. In some embodiments, the second sub-interval has a duration that is at least about 40%, at least about 50%, at least about 60%, at least about 70%, at least about 75%, at least about 80%, at least about 85%, or at least about 90% of the duration of the exposure interval. In certain embodiments, the second sub-interval has a duration that is in a range of 40% to 60%, 40% to 70%, 40% to 80%, 40% to 90%, 50% to 70%, 50% to 80%, 50% to 90%, 60% to 80%, 60% to 90%, 70% to 90%, or 80% to 90% of the duration of the exposure interval.

In some embodiments, the second wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In some embodiments, the second wavelength is approximately 380 nm, 400 nm, 450 nm, 470 nm, 495 nm, 565 nm, 610 nm, 625 nm, or 750 nm. In certain embodiments, light at the second wavelength is emitted from the second light source at an intensity (e.g., at the sample plane) of at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm². In certain embodiments, light at the second wavelength is emitted from the second light source at an intensity (e.g., at the sample plane) in a range from 2 W/cm² to 5 W/cm², 2 W/cm² to 10 W/cm², 3 W/cm² to 5 W/cm², 3 W/cm² to 10 W/cm², 5 W/cm² to 10 W/cm², or 8 W/cm² to 10 W/cm².

In some embodiments, the method comprises controlling the first light source and the second light source such that the sample is not illuminated by the first light source and/or the second light source during a third sub-interval of the exposure interval between the first sub-interval and the second sub-interval. In some embodiments, the sample is not illuminated by the first light source and the second light source during the third sub-interval. In some embodiments, the sample is not illuminated by the first light source but is illuminated by the second light source during the third sub-interval. In some embodiments, the third sub-interval has a duration of about 100 μs or less, about 80 μs or less, about 50 μs or less, about 40 μs or less, about 30 μs or less, about 20 μs or less, about 10 μs or less, or about 5 μs or less. In some embodiments, the third sub-interval has a duration of 5 μs to 10 μs, 5 μs to 20 μs, 5 μs to 30 μs, 5 μs to 40 μs, 5 μs to 50 μs, 5 μs to 80 μs, 10 μs to 20 μs, 10 μs to 30 μs, 10 μs to 40 μs, 10 μs to 50 μs, 10 μs to 80 μs, 20 μs to 40 μs, 20 μs to 50 μs, 20 μs to 80 μs, 20 μs to 100 μs, 50 μs to 80 μs, 50 μs to 100 μs, or 80 μs to 100 μs.

In some embodiments, the method of projecting two or more spatial patterns of illumination at two or more wavelengths concurrently comprises controlling the spatial light modulator to project two or more spatial patterns of light at the same wavelength onto the sample during the same sub-interval. In certain embodiments, a sub-interval described herein comprises a plurality of sub-sub-intervals. In certain embodiments, a spatial pattern described herein (e.g., a first spatial pattern, a second spatial pattern) comprises a plurality of sub-patterns. The plurality of sub-patterns may comprise at least 2 sub-patterns, at least 5 sub-patterns, at least 10 sub-patterns, at least 20 sub-patterns, at least 50 sub-patterns, at least 80 sub-patterns, at least 100 sub-patterns, at least 200 sub-patterns, or at least 500 sub-patterns. In certain embodiments, the plurality of sub-patterns comprises between 2 and 5 sub-patterns, 2 and 10 sub-patterns, 2 and 20 sub-patterns, 2 and 50 sub-patterns, 2 and 100 sub-patterns, 2 and 200 sub-patterns, 2 and 500 sub-patterns, 10 and 20 sub-patterns, 10 and 50 sub-patterns, 10 and 100 sub-patterns, 10 and 200 sub-patterns, 10 and 500 sub-patterns, 20 and 50 sub-patterns, 20 and 100 sub-patterns, 20 and 200 sub-patterns, 20 and 500 sub-patterns, 50 and 100 sub-patterns, 50 and 200 sub-patterns, 50 and 500 sub-patterns, 100 and 200 sub-patterns, 100 and 500 sub-patterns, or 200 and 500 sub-patterns. In some embodiments, the method comprises controlling the spatial light modulator to project a first sub-pattern at a wavelength (e.g., the first wavelength, the second wavelength) during a first sub-sub-interval of a sub-interval. In some embodiments, the method further comprises controlling the spatial light modulator to project a second sub-pattern at the wavelength during a second sub-sub-interval of the sub-interval. In some embodiments, the method comprises controlling the spatial light modulator to project each sub-pattern of a spatial pattern during a separate sub-sub-interval of a sub-interval. In some embodiments, two or more sub-patterns are orthogonal or near orthogonal (e.g., have a correlation coefficient between the two sub-patterns of 0.25 or less). In certain embodiments, all sub-patterns are orthogonal or near-orthogonal to each other.

In some embodiments, the method of projecting two or more spatial patterns of illumination at two or more wavelengths concurrently further comprises controlling a third light source to emit light at a third wavelength during a fourth sub-interval of the exposure interval. In some embodiments, the third wavelength is different from the first wavelength and the second wavelength. In some embodiments, the method comprises controlling the spatial light modulator to project a third spatial pattern of light at the third wavelength onto the sample during the fourth sub-interval. In some embodiments, the fourth sub-interval does not overlap the first, second, or third sub-intervals. The fourth sub-interval may occur before or after the first sub-interval. The fourth sub-interval may occur before or after the second sub-interval. In some embodiments, the fourth sub-interval has a duration of 1 ms or less, 800 μs or less, 500 μs or less, 250 μs or less, 200 μs or less, 150 μs or less, 100 μs or less, 50 us or less, or 20 μs or less. In some embodiments, the fourth sub-interval has a duration in a range from 20 μs to 50 μs, 20 μs to 100 μs, 20 μs to 200 μs, 20 μs to 500 μs, 20 μs to 1 ms, 50 μs to 100 μs, 50 μs to 200 μs, 50 μs to 500 μs, 50 μs to 1 ms, 100 μs to 200 μs, 100 μs to 500 μs, 100 μs to 1 ms, 200 μs to 500 μs, 200 μs to 1 ms, or 500 μs to 1 ms. In some embodiments, the fourth sub-interval has a duration that is about 50% or less, 40% or less, 30% or less, 20% or less, or 10% or less of the duration of the exposure interval. In certain embodiments, the fourth sub-interval has a duration that is in a range of 10% to 20%, 10% to 30%, 10% to 40%, 10% to 50%, 20% to 40%, 20% to 50%, 30% to 50%, or 40% to 50% of the duration of the exposure interval.

In some embodiments, the third wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In some embodiments, the third wavelength is approximately 380 nm, 400 nm, 450 nm, 470 nm, 495 nm, 565 nm, 610 nm, 625 nm, or 750 nm. In certain embodiments, light at the third wavelength is emitted from the third light source at an intensity (e.g., at the sample plane) of at least 2 W/cm², at least 3 W/cm², at least 4 W/cm², at least 5 W/cm², at least 6 W/cm², at least 7 W/cm², at least 8 W/cm², at least 9 W/cm², or at least 10 W/cm². In certain embodiments, light at the third wavelength is emitted from the third light source at an intensity (e.g., at the sample plane) in a range from 2 W/cm² to 5 W/cm², 2 W/cm² to 10 W/cm², 3 W/cm² to 5 W/cm², 3 W/cm² to 10 W/cm², 5 W/cm² to 10 W/cm², or 8 W/cm² to 10 W/cm².

In some embodiments, the method of projecting two or more spatial patterns of illumination at two or more wavelengths concurrently comprises controlling four or more light sources and controlling the spatial light modulator to project four or more spatial patterns of light at four or more wavelengths onto the sample. In some embodiments, an imaging system comprises at least four, at least five, at least ten, or at least twenty light sources. In certain embodiments, an imaging system comprises 4 to 10, 4 to 20, 5 to 10, 5 to 20, or 10 to 20 light sources. In some cases, each light source may be associated with a distinct sequence of one or more spatial patterns to be projected onto the sample.

Some aspects are directed to methods of spatial registration of spatial light modulator coordinates to imaging device coordinates. In order to specify patterns of illumination on a spatial light modulator that will accurately illuminate portions of a sample for imaging by an imaging device, mapping of pixel coordinates on the spatial light modulator to pixel coordinates on the imaging device is needed.

In some embodiments, a method of spatial registration comprises using a spatial light modulator to sequentially project a plurality of spatial patterns onto a sample (e.g., a fluorescent test slide), where each spatial pattern is configured to illuminate a single pixel (or, in some cases, a few pixels). As an illustrative example, the plurality of spatial patterns may comprise four spatial patterns, where each spatial pattern is configured to illuminate one of four vertices of a square. Each illuminated pixel may be associated with spatial light modulator coordinates. In some embodiments, the method further comprises using an imaging device to obtain one image for each spatial pattern. For example, in the illustrative example involving four spatial patterns, four images may be obtained, with each image displaying an illuminated pixel. The centroid of each pixel in imaging device coordinates may be obtained. Each illuminated pixel may therefore be associated with both spatial light modulator coordinates and imaging device coordinates. In some embodiments, the method comprises performing an affine transformation, M, of spatial light modulator coordinates to imaging device coordinates. In certain embodiments, the method comprises calculating the inverse transformation, M⁻¹, to map imaging device coordinates to spatial light modulator coordinates.

In some cases, the method of spatial registration further comprises one or more nonlinear mapping steps (e.g., to correct for optical distortions or otherwise increase mapping accuracy). In some embodiments, the method comprises defining an array of spots (e.g., a 10×10 grid of spots) in imaging device coordinates. In some embodiments, the method comprises mapping the array of spots to spatial light modulator coordinates using the transformation M⁻¹. In some cases, the method comprises projecting the mapped array onto a sample (e.g., a fluorescent test slide) using the spatial light modulator. An image of the array may be obtained using the imaging device, and the centroid of each illuminated spot may be obtained in imaging device coordinates. A non-linear transformation may be computed to accurately map the spatial light modulator coordinates to these imaging device coordinates. The transform and its inverse may then be used for future spatial light modulator-to-imaging device pixel mappings.

Some aspects are directed to methods of imaging a sample. In some embodiments, the sample is a biological sample. The biological sample may be obtained from a human or a non-human animal (e.g., a mouse, a rat, a pig, a rabbit, a monkey). In certain embodiments, the biological sample comprises an in vitro tissue sample. In certain embodiments, the biological sample comprises in vivo tissue.

In some embodiments, the biological sample comprises a plurality of cells comprising one or more fluorescent reporters configured to emit fluorescence in response to excitation light. Non-limiting examples of fluorescent reporters include voltage reporters, calcium reporters, and cAMP reporters. Examples of voltage reporters include, but are not limited to, QuasAr and Voltron proteins. Examples of calcium reporters include, but are not limited to, GCaMP and red-shifted genetically encoded Ca²⁺ indicators. Examples of cAMP reporters include, but are not limited to, the Pink Flamindo protein.

In some embodiments, the plurality of cells comprise one or more light-activated molecules configured to respond to activation light (also referred to as a stimulus). In certain embodiments, the one or more light-activated molecules comprise a light-activated protein. Non-limiting examples of light-activated proteins include channelrhodopsins and adenylyl cyclases. A channelrhodopsin generally refers to a rhodopsin that functions as a light-gated ion channel (e.g., upon exposure to activation light, electrical activity in a cell may be activated or suppressed). Non-limiting examples of channelrhodopsins include channelrhodopsin-1 (ChR1), channelrhodopsin-2 (ChR2), Volvox-derived ChR1 (VChR1), step-function opsins (SFOs), and variants thereof. An adenylyl cyclase generally refers to an enzyme that catalyzes the formation of cyclic adenosine monophosphate (cAMP). For example, a bacterial photoactivated adenylyl cyclase (bPAC) converts a blue light stimulus into production of cAMP. In some instances, the light-activated protein mimics morphogens involved in embryonic development and/or triggers growth or assembly of cytoskeletal elements. In certain embodiments, the one or more light-activated molecules comprise a light-activated organic compound. Non-limiting examples of light-activated organic compounds include a photo-caged tetrazine compound, a photocrosslinker, and a compound comprising an azobenzene moiety. In some instances, for example, a photo-caged tetrazine compound can initiate a click chemistry reaction when illuminated. In some instances, a photocrosslinker can immobilize molecules or cells when illuminated. In some instances, a compound comprising an azobenzene moiety can undergo photoisomerization (e.g., between trans and cis isomers) when illuminated.

The cells of the plurality of cells may be any type of cells. In some embodiments, the cells of the plurality of cells are neurons. In some embodiments, the cells of the plurality of cells are myocytes (e.g., cardiomyocytes, skeletal myocytes). In some embodiments, the cells of the plurality of cells are embryonic stem cells.

In some embodiments, a method of imaging a sample comprises illuminating the sample with a first sequence of spatial patterns (e.g., two-dimensional spatial patterns) using light of a first wavelength. In certain embodiments, illuminating the sample with the first sequence of spatial patterns comprises controlling a first light source to emit light of the first wavelength during a first interval. In certain embodiments, illuminating the sample with the first sequence of spatial patterns comprises controlling a spatial light modulator (e.g., a DMD) to project the first sequence of spatial patterns onto the sample during the first interval. In some embodiments, the method of imaging the sample further comprises capturing image data based on light of a second wavelength emitted by the sample (e.g., fluorescent reporters expressed in the sample) using an imaging device during the first interval. In some embodiments, the image data comprises a plurality of frames. In certain embodiments, each frame of the plurality of frames is associated with a respective spatial pattern of the first sequence of spatial patterns.

In some embodiments, each spatial pattern of the first sequence of spatial patterns is configured such that neighboring locations within the sample are illuminated with orthogonal functions of intensity. In some embodiments, each spatial pattern of the first series of spatial patterns is a binary spatial pattern. In some embodiments, each spatial pattern of the first sequence of spatial patterns is orthogonal or near orthogonal to the other spatial patterns of the first sequence. Two spatial patterns are said to be “near orthogonal” when a correlation coefficient between the two spatial patterns is 0.25 or less. In certain embodiments, even when the spatial patterns are not orthogonal, some of the same benefits of using orthogonal spatial patterns may be achieved by analyzing orthogonal components of the image data.

In some embodiments, the first sequence of spatial patterns is generated using a Hadamard matrix, as described in International Patent Application No. PCT/US2018/015517, entitled “Widefield, High-Speed Optical Sectioning,” and filed Jan. 26, 2018, which is hereby incorporated by reference. A Hadamard matrix, H, of size m is a binary square matrix with elements {−1,1} that fulfills H^{T}H=mIₘ, where Iₘ is the identity matrix of size m; its normalized form has value 1 in the first column and first row. Illumination intensities may not be negative, so the projected intensity patterns may be given by P=(H′+1)/2, where H′=H [1, . . . , m; m−n+1, . . . , m] is a submatrix that is an incomplete orthogonal basis given by the last n columns of a normalized Hadamard matrix, with n<m. The illumination patterns P thus may have binary values {0,1} corresponding to spatial light modulator positions OFF and ON, respectively. In some embodiments, each location may be illuminated with a positive temporal function orthogonal to the other designed Hadamard sequences, as verified by PTH′=Iₙ*m/2. In some examples, for a given number of locations, a Hadamard matrix provides a set of binary orthogonal functions. In some embodiments, the set is the shortest possible set. In some embodiments, to arrange n codes in P into illumination patterns, m=n+1 images are defined assigning code k_{i,j}∈{1 . . . n} to spatial modulator pixel (i,j), as k_{i,j}=mod (i*q+j,n)+1, where q is an offset parameter that maximizes spatial separation of repeated codes.

In some embodiments, the first sequence of spatial patterns is generated by tiling a Hadamard matrix to fill the pixels of the spatial light modulator. In some embodiments, the number of elements in the Hadamard matrix determines the number of patterns in the sequence. In certain embodiments, a Hadamard matrix smaller than the total number of pixels of the spatial light modulator may be used, resulting in a sequence with non-orthogonal patterns. However, the patterns in the sequence may be near orthogonal and the negative effects of any non-orthogonality can be reduced by the fact that only nearby portions of the sample will scatter light that affects the quality of the image, and the portions of the sample near any particular point are likely to be illuminated by an orthogonal sequence.

In some embodiments, the first sequence of spatial patterns comprises at least 10 spatial patterns, at least 20 spatial patterns, at least 50 spatial patterns, at least 80 spatial patterns, or at least 100 spatial patterns. In some embodiments, the first sequence of spatial patterns comprises 10 to 20 spatial patterns, 10 to 50 spatial patterns, 10 to 80 spatial patterns, 10 to 100 spatial patterns, 20 to 50 spatial patterns, 20 to 80 spatial patterns, 20 to 100 spatial patterns, 50 to 80 spatial patterns, 50 to 100 spatial patterns, or 80 to 100 spatial patterns. In some embodiments, each spatial pattern of the first sequence of spatial patterns is repeatedly projected onto the sample. In certain embodiments, each spatial pattern of the first sequence of spatial patterns is projected onto the sample at least 5 times, at least 10 times, at least 20 times, at least 50 times, at least 80 times, or at least 100 times. In some embodiments, each spatial pattern of the first sequence of spatial patterns is projected onto the sample 5 to 10 times, 5 to 20 times, 5 to 50 times, 5 to 80 times, 5 to 100 times, 10 to 20 times, 10 to 50 times, 10 to 80 times, 10 to 100 times, 20 to 50 times, 20 to 80 times, 20 to 100 times, 50 to 80 times, 50 to 100 times, or 80 to 100 times. In some cases, images from the repeated illuminations may be reconstructed and averaged to yield a presegmentation image.

The first wavelength may be any suitable wavelength. In certain embodiments, the first wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In certain embodiments, the first wavelength is in a range from 590 nm to 625 nm. In certain embodiments, the first wavelength is approximately 380 nm, 400 nm, 450 nm, 470 nm, 495 nm, 510 nm, 548 nm, 565 nm, 610 nm, 625 nm, 647 nm, 690 nm, or 750 nm.

The second wavelength may be any suitable wavelength. In certain embodiments, the second wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In certain embodiments, the second wavelength is approximately 380 nm, 400 nm, 450 nm, 470 nm, 495 nm, 525 nm, 565 nm, 610 nm, 625 nm, 670 nm, 705 nm, or 750 nm.

In some embodiments, the method of imaging the sample comprises constructing a second spatial pattern. In certain embodiments, the second spatial pattern is configured to provide targeted illumination of substantially all cells expressing the one or more fluorescent reporters. By avoiding illumination of interstitial parts of the sample, the targeted illumination may substantially decrease out-of-focus background fluorescence.

In certain embodiments, constructing the second spatial pattern comprises filtering a presegmentation image. A non-limiting example of a suitable filter is a Gaussian filter. In certain embodiments, constructing the second spatial pattern comprises segmenting the filtered or unfiltered presegmentation image using a segmentation algorithm. In certain embodiments, the segmentation algorithm is a watershed segmentation algorithm. In some such embodiments, watershed regions comprising fewer than a threshold number of pixels (e.g., 10 pixels or less, 5 pixels or less, 2 pixels or less) may be discarded. In some embodiments, a putative cell within each non-discarded watershed region may be located by identifying the maximum intensity pixel. In some cases, watershed regions most correlated with the filter (e.g., the Gaussian filter) are selected as containing cells expressing the one or more fluorescent reporters, where correlation is calculated using the unfiltered presegmentation image. In some cases, other segmentation algorithms (e.g., clustering algorithms, region-based algorithms, thresholding algorithms, edge-based algorithms) may be used to identify and locate cells in the presegmentation image.

In some embodiments, constructing the second spatial pattern comprises generating an illumination mask for each selected cell. In some embodiments, the illumination mask is substantially circular, substantially elliptical, substantially square, substantially rectangular, or substantially hexagonal. The size of the illumination masks may vary based on the type of cell being imaged. In certain embodiments, the illumination mask for each cell has a largest dimension (e.g., diameter) of at least 5 μm, at least 10 μm, at least 20 μm, at least 30 μm, at least 40 μm, at least 50 μm, at least 80 μm, at least 100 μm, at least 150 μm, at least 200 μm, at least 500 μm, or at least 1 mm. In certain embodiments, the illumination mask for each cell has a largest dimension in a range from 5 to 20 μm, 5 to 50 μm, 5 to 100 μm, 5 to 150 μm, 5 to 200 μm, 5 to 500 μm, 5 μm to 1 mm, 20 to 50 μm, 20 to 100 μm, 20 to 150 μm, 20 to 200 μm, 20 to 500 μm, 20 μm to 1 mm, 50 to 100 μm, 50 to 150 μm, 50 to 200 μm, 50 to 500 μm, 50 μm to 1 mm, 100 to 150 μm, 100 to 200 μm, 100 to 500 μm, 100 μm to 1 mm, 200 to 500 μm, 200 μm to 1 mm, or 500 μm to 1 mm.

In some embodiments, the method of imaging the sample comprises constructing a third sequence of spatial patterns. In some cases, the third sequence of spatial patterns permits targeted illumination of certain subsets of cells to be stimulated. By sequentially stimulating subsets of cells and recording the responses of those and other cells, the functional connections between cells may be mapped.

In some embodiments, each spatial pattern of the third sequence of spatial patterns comprises illumination masks for a subset of cells of the second spatial pattern. In certain embodiments, each cell of a spatial pattern is separated from other cells of the spatial pattern by a minimum distance. In some cases, ensuring a certain minimum distance between cells may allow an influence on one cell (e.g., a putative post-synaptic cell) to be attributed with relatively high probability to a specific cell (e.g., a specific pre-synaptic cell). In certain embodiments, the minimum distance is at least 100 μm, at least 200 μm, at least 300 μm, at least 400 μm, at least 500 μm, at least 800 μm, at least 1 mm, or at least 1.5 mm. In certain embodiments, the minimum distance is in a range from 100 μm to 200 μm, 100 μm to 500 μm, 100 μm to 800 μm, 100 μm to 1 mm, 100 μm to 1.5 mm, 200 μm to 500 μm, 200 μm to 800 μm, 200 μm to 1 mm, 200 μm to 1.5 mm, 500 μm to 800 μm, 500 μm to 1 mm, 500 μm to 1.5 mm, 800 μm to 1 mm, or 800 μm to 1.5 mm.

In some embodiments, constructing the third sequence of spatial patterns comprises generating an illumination mask for each cell of a subset of cells. In some embodiments, the illumination mask is substantially circular, substantially elliptical, substantially square, substantially rectangular, or substantially hexagonal. The size of the illumination masks may vary based on the type of cell being imaged. In certain embodiments, the illumination mask for each cell has a largest dimension (e.g., diameter) of at least 5 μm, at least 10 μm, at least 20 μm, at least 30 μm, at least 40 μm, at least 50 μm, at least 80 μm, at least 100 μm, at least 150 μm, at least 200 μm, at least 500 μm, or at least 1 mm. In certain embodiments, the illumination mask for each cell has a largest dimension in a range from 5 to 20 μm, 5 to 50 μm, 5 to 100 μm, 5 to 150 μm, 5 to 200 μm, 5 to 500 μm, 5 μm to 1 mm, 20 to 50 μm, 20 to 100 μm, 20 to 150 μm, 20 to 200 μm, 20 to 500 μm, 20 μm to 1 mm, 50 to 100 μm, 50 to 150 μm, 50 to 200 μm, 50 to 500 μm, 50 μm to 1 mm, 100 to 150 μm, 100 to 200 μm, 100 to 500 μm, 100 μm to 1 mm, 200 to 500 μm, 200 μm to 1 mm, or 500 μm to 1 mm.

In some embodiments, the third sequence of spatial patterns comprises at least 2 spatial patterns, at least 5 spatial patterns, at least 10 spatial patterns, at least 15 spatial patterns, at least 20 spatial patterns, at least 50 spatial patterns, at least 100 spatial patterns, at least 150 spatial patterns, at least 200 spatial patterns, at least 500 spatial patterns, or at least 1000 spatial patterns. In certain embodiments, the third sequence of spatial patterns comprises 2 to 5 spatial patterns, 2 to 10 spatial patterns, 2 to 20 spatial patterns, 2 to 50 spatial patterns, 2 to 100 spatial patterns, 2 to 200 spatial patterns, 2 to 500 spatial patterns, 2 to 1000 spatial patterns, 10 to 20 spatial patterns, 10 to 50 spatial patterns, 10 to 100 spatial patterns, 10 to 200 spatial patterns, 10 to 500 spatial patterns, 10 to 1000 spatial patterns, 50 to 100 spatial patterns, 50 to 200 spatial patterns, 50 to 500 spatial patterns, 50 to 1000 spatial patterns, 100 to 500 spatial patterns, 100 to 1000 spatial patterns, or 500 to 1000 spatial patterns. In some embodiments, two or more spatial patterns of the third sequence of spatial patterns are orthogonal or near orthogonal. In certain embodiments, all spatial patterns of the third sequence of spatial patterns are orthogonal or near orthogonal to each other.

In some embodiments, the method of imaging the sample comprises illuminating the sample with the second spatial pattern at the first wavelength and one or more spatial patterns of the third sequence of spatial patterns at a third wavelength during a first stimulus period. In some embodiments, illumination with the second spatial pattern is alternated with illumination with the one or more spatial patterns of the third sequence of spatial patterns at a rate higher than a frame rate of the imaging device. That is, the second spatial pattern and one or more spatial patterns of the third sequence of spatial patterns are interleaved with sub-time frame resolution.

In some embodiments, the method comprises initiating an exposure of the imaging device for an exposure interval, wherein the exposure interval comprises a plurality of sub-intervals. In certain embodiments, the exposure interval has a duration of at least 0.5 ms, at least 1 ms, at least 2 ms, at least 3 ms, at least 4 ms, at least 5 ms, at least 10 ms, at least 15 ms, or at least 20 ms. In certain embodiments, the exposure interval has a duration of 0.5 to 1 ms, 0.5 to 2 ms, 0.5 to 5 ms, 0.5 to 10 ms, 0.5 to 15 ms, 0.5 to 20 ms, 1 to 2 ms, 1 to 5 ms, 1 to 10 ms, 1 to 15 ms, 1 to 20 ms, 5 to 10 ms, 5 to 15 ms, 5 to 20 ms, or 10 to 20 ms.

In some embodiments, the method comprises controlling the first light source to emit light at the first wavelength during a first sub-interval of the exposure interval. In some embodiments, the method comprises controlling the spatial light modulator to project the second spatial pattern onto the sample during the first sub-interval. In some embodiments, the first sub-interval has a duration of about 10 ms or less, 5 ms or less, 2 ms or less, 1 ms or less, 500 μs or less, 200 μs or less, 100 μs or less, 50 μs or less, 20 μs or less, or 10 μs or less. In certain embodiments, the first sub-interval has a duration of 10 μs to 50 μs, 10 μs to 100 μs, 10 μs to 500 μs, 10 μs to 1 ms, 10 μs to 5 ms, 10 μs to 10 ms, 50 μs to 100 μs, 50 μs to 500 μs, 50 μs to 1 ms, 50 μs to 5 ms, 50 μs to 10 ms, 100 μs to 500 μs, 100 μs to 1 ms, 100 μs to 5 ms, 100 μs to 10 ms, 500 μs to 1 ms, 500 μs to 5 ms, 500 μs to 10 ms, 1 ms to 5 ms, 1 ms to 10 ms, or 5 ms to 10 ms. In some embodiments, the first light source emits light at the first wavelength at an intensity (e.g., at the sample plane) of at least 2 mW/mm², at least 3 mW/mm², at least 4 mW/mm², at least 5 mW/mm², at least 10 mW/mm², at least 15 mW/mm², at least 20 mW/mm², at least 50 mW/mm², at least 80 mW/mm², or at least 100 mW/mm². In certain embodiments, the first light source emits light at the first wavelength at an intensity (e.g., at the sample plane) of 2 to 5 mW/mm², 2 to 10 mW/mm², 2 to 15 mW/mm², 2 to 20 mW/mm², 2 to 50 mW/mm², 2 to 80 mW/mm², 2 to 100 mW/mm², 5 to 10 mW/mm², 5 to 15 mW/mm², 5 to 20 mW/mm², 5 to 50 mW/mm², 5 to 80 mW/mm², 5 to 100 mW/mm², 10 to 20 mW/mm², 10 to 50 mW/mm², 10 to 80 mW/mm², 10 to 100 mW/mm², 15 to 20 mW/mm², 20 to 50 mW/mm², 20 to 80 mW/mm², 20 to 100 mW/mm², 50 to 80 mW/mm², 50 to 100 mW/mm², or 80 to 100 mW/mm².

In some embodiments, the method of imaging the sample comprises controlling the second light source to emit light of a third wavelength during a second sub-interval of the exposure interval, wherein the second sub-interval is different from the first sub-interval. In some embodiments, the method comprises controlling the spatial light modulator to project one or more spatial patterns of the third sequence of spatial patterns onto the sample during the second sub-interval. In some embodiments, the second sub-interval does not overlap with the first sub-interval. In some embodiments, the second sub-interval has a duration of about 1 ms or less, 500 μs or less, 200 μs or less, 100 μs or less, 50 μs or less, 20 μs or less, or 10 us or less. In certain embodiments, the second sub-interval has a duration of 10 μs to 50 μs, 10 μs to 100 μs, 10 μs to 500 μs, 10 μs to 1 ms, 50 μs to 100 μs, 50 μs to 500 μs, 50 μs to 1 ms, 100 μs to 500 μs, 100 μs to 1 ms, or 500 μs to 1 ms.

The third wavelength may be any suitable wavelength. In certain embodiments, the third wavelength is in a range from 380 nm to 450 nm, 400 nm to 700 nm, 450 nm to 495 nm, 495 nm to 565 nm, 565 nm to 590 nm, 590 nm to 625 nm, or 625 nm to 750 nm. In certain embodiments, the third wavelength is in a range from 450 nm to 495 nm. In certain embodiments, the third wavelength is approximately 380 nm, 400 nm, 450 nm, 470 nm, 473 nm, 480 nm, 495 nm, 510 nm, 548 nm, 565 nm, 610 nm, 625 nm, 647 nm, 690 nm, or 750 nm.

In some embodiments, the second light source emits light of the third wavelength at an intensity (e.g., at the sample plane) of at least 2 mW/mm², at least 3 mW/mm², at least 4 mW/mm², at least 5 mW/mm², at least 10 mW/mm², at least 15 mW/mm², at least 20 mW/mm², at least 50 mW/mm², at least 80 mW/mm², or at least 100 mW/mm². In certain embodiments, the second light source emits light of the third wavelength at an intensity (e.g., at the sample plane) of 2 to 5 mW/mm², 2 to 10 mW/mm², 2 to 15 mW/mm², 2 to 20 mW/mm², 2 to 50 mW/mm², 2 to 80 mW/mm², 2 to 100 mW/mm², 5 to 10 mW/mm², 5 to 15 mW/mm², 5 to 20 mW/mm², 5 to 50 mW/mm², 5 to 80 mW/mm², 5 to 100 mW/mm², 10 to 20 mW/mm², 10 to 50 mW/mm², 10 to 80 mW/mm², 10 to 100 mW/mm², 15 to 20 mW/mm², 20 to 50 mW/mm², 20 to 80 mW/mm², or 20 to 100 mW/mm².

In some embodiments, two or more spatial patterns of the third sequence of spatial patterns are projected onto the sample during the second sub-interval. In certain embodiments, the second sub-interval comprises a plurality of sub-sub-intervals. In some cases, a first spatial pattern of the third sequence of spatial patterns may be projected onto the sample during a first sub-sub-interval of the second sub-interval and a second spatial pattern of the third sequence of spatial patterns may be projected onto the sample during a second sub-sub-interval of the second sub-interval.

In some embodiments, the method of imaging the sample comprises controlling the first light source and a second light source such that the sample is not illuminated by the first light source and/or the second light source during a third sub-interval between the first sub-interval and the second sub-interval. In some embodiments, the third sub-interval has a duration of about 100 μs or less, about 80 μs or less, about 50 μs or less, about 40 μs or less, about 30 μs or less, about 20 μs or less, about 10 μs or less, or about 5 μs or less. In some embodiments, the third sub-interval has a duration of 5 to 10 μs, 5 to 20 μs, 5 to 50 μs, 5 to 80 μs, 5 to 100 μs, 10 to 20 μs, 10 to 50 μs, 10 to 80 μs, 10 to 100 μs, 20 to 50 μs, 20 to 80 μs, 20 to 100 μs, 50 to 80 μs, 50 to 100 μs, or 80 to 100 μs.

In some embodiments, one or more (or, in some cases, all) spatial patterns of the third sequence of spatial patterns are repeatedly projected onto the sample by the spatial light modulator. In certain embodiments, for example, a first spatial pattern of the third sequence of spatial patterns is repeatedly alternated with the second spatial pattern for a first stimulation interval. In certain embodiments, a second spatial pattern of the third sequence of spatial patterns is repeatedly alternated with the second spatial pattern for a second stimulation interval. In some cases, the first and second interval do not overlap. In some cases, each spatial pattern of the third sequence of spatial patterns may be repeatedly alternated with the second spatial pattern for an interval until all cells have been stimulated. In some cases, repeating each spatial pattern may permit later construction of a stimulus-triggered average response in each putative responsive cell (e.g., putative postsynaptic cell). In some cases, each cell may be stimulated at least 10, at least 20, at least 50, at least 100, at least 200, at least 500, at least 1000, at least 2000, at least 5000, at least 10,000, at least 20,000, or at least 50,000 times. In some cases, each cell may be stimulated 10-20, 10-50, 10-100, 10-200, 10-500, 10-1000, 10-2000, 10-5000, 10-10000, 10-20000, or 10-50000 times. In some cases, a stimulation interval has a duration of at least 500 μs, at least 1 ms, at least 1.25 ms, at least 1.5 ms, at least 2 ms, at least 5 ms, at least 10 ms, at least 20 ms, at least 50 ms, at least 100 ms, at least 200 ms, at least 500 ms, or at least 1 s. In some cases, a stimulation interval has a duration in a range from 500 μs to 1 ms, 500 μs to 1.5 ms, 500 μs to 2 ms, 500 μs to 5 ms, 500 μs to 10 ms, 500 μs to 20 ms, 500 μs to 50 ms, 500 μs to 100 ms, 500 μs to 200 ms, 500 μs to 500 ms, 500 μs to 1 s, 1 ms to 1.5 ms, 1 ms to 2 ms, 1 ms to 5 ms, 1 ms to 10 ms, 1 ms to 20 ms, 1 ms to 50 ms, 1 ms to 100 ms, 1 ms to 200 ms, 1 ms to 500 ms, 1 ms to 1 s, 10 ms to 50 ms, 10 ms to 100 ms, 10 ms to 200 ms, 10 ms to 500 ms, 10 ms to 1 s, 100 ms to 200 ms, 100 ms to 500 ms, 100 ms to 1 s, or 500 ms to 1 s. In some embodiments, one or more stimulation intervals are followed by one or more rest intervals. During a rest interval, illumination with light at the third wavelength may be turned off while illumination with light at the first wavelength may be maintained. In some cases, during a rest interval, illumination with light at the first wavelength and at the third wavelength may be turned off. In some cases, a rest period has a duration of at least about 1 ms, 2 ms, 5 ms, 10 ms, 20 ms, 50 ms, 100 ms, 200 ms, 500 ms, 1 s, 2 s, 5 s, 10 s, 20 s, or 50 s.

In some cases, the method of imaging the sample comprises correcting for mechanical drift of cells during imaging. In some cases, a Hadamard image may be obtained at regular intervals (e.g., every 6 minutes). Drifts in lateral directions may be corrected using displacement vectors. In some embodiments, the displacement vectors may be calculated by the Farneback optical flow algorithm. Drifts in the vertical direction may be corrected using displacements calculated by correlating the fast Fourier transform of the Hadamard image with that of a 6 μm deep confocal z-stack (3 μm resolution). The most correlated depth may be found by fitting the correlation with a quadratic polynomial.

U.S. Provisional Application No. 63/460,250, filed Apr. 18, 2023, is hereby incorporated by reference in its entirety.

The following examples are intended to illustrate certain embodiments of the present disclosure, but do not exemplify the full scope of the disclosure.

### Example 1

In this Example, a system for conveying high-intensity orange LED illumination to a sample was developed and built.

Commercially available orange light-emitting diodes (e.g., Osram GY CS8PM1.23-KPKR-36) are specified to produce 160 mW of light at a current of 350 mA and a temperature of 25° C. Voltage imaging generally requires approximately 500 mW of light, corresponding to 3-fold greater optical power. Due to the requirement to focus the light on a small area of the sample (e.g., a diameter less than or equal to 2 mm), the greater optical power should come from a single LED producing more light instead of multiple LEDs arranged in parallel.

The higher optical power was achieved by (a) cooling the LED to −30° C., and (b) driving it with a higher current of 1 A. Cooling the LED increased the light output by a factor of 1.6, and increasing the drive current to 1 A increased the light output by another factor of 2, for a total increase by a factor of 3.2. This increase was sufficient to achieve the 500 mW target power. The electrical power dissipation was 3 W.

To keep the LED at −30° C. while simultaneously dissipating the 3 W of electrical power required a specialized cooling and heat-sink design. To cool the LED, a Peltier thermoelectric cooler (Sheetak SKHC1-199-16-T100-SS-TF00-ALO) backed by a water-cooled heat sink (EK-Quantum Velocity) was used.

Since the LED was below the dew point under ambient conditions, an inert-gas enclosure was designed to prevent condensation. The enclosure comprised an acrylic cylinder affixed to the heat sink with a gas-tight connection and a 50.8 mm diameter optical window at the front.

Due to the wide angular spread of the LED illumination, the LED light would have diverged too much by the time it passed the window. A first collimating lens was therefore mounted inside the inert-gas chamber. This lens comprised an aspheric condenser lens, Ø1″, focal length 16 mm, with an anti-reflective coating from 350-700 nm.

Due to the broad emission spectrum of the LED, a portion of the LED light would have leaked through the emission filters and contaminated the fluorescence signal on the camera. To avoid this problem, an excitation filter (Chroma ET580/25x-25mmR) which removed the long-wavelength component of the LED illumination was also mounted inside the inert gas chamber.

Outside the chamber, a plano-convex lens, Ø2″, focal length 10 cm (Thorlabs LA1050-A-ML) was placed. This lens was positioned 10 cm from the LED to further collimate the LED emission.

For blue illumination, a custom housing with a water-cooled heatsink was also used. Inside the LED housing, the LED emission was collimated with an aspheric condenser lens, Ø1″, focal length 16 mm, with an anti-reflective coating from 350-700 nm followed by a Ø1″ 10 cm focal length achromat.

The light from the orange and blue LEDs was combined on a custom 60×85 mm long-pass dichroic with a cutoff wavelength of 580 nm. The light from both LEDs was then focused onto the digital micromirror device via a plano-convex lens (Thorlabs LA1238-A, Ø75.0 mm, f=100 mm, AR Coating: 350-700 nm).

### Example 2

In this Example, a system for imaging with high numerical aperture and large field of view was developed and built.

Previous wide-area microscope designs provided a magnification of M=2.7× and a numerical aperture of NA=0.5. The imaging system was based around a nominal 2× magnification objective (MVPLAPO 2XC), with a 10 cm back-focal length. In a previous wide-area optical system, the image was formed by a 135 mm camera lens (ZEISS MILVUS 135 mm f/2) for a total system magnification of 2.7×. The light was collected on a scientific CMOS (sCMOS) camera (Hamamatsu Orca Flash v4.2).

Voltage imaging generally requires a frame rate greater than or equal to 400 Hz. Since scientific CMOS cameras are limited in the number of rows they can read out at this speed, increasing the field of view generally requires decreasing the magnification. In this Example, the 135 mm Zeiss camera lens of the previous wide-area optical system was replaced by a 50 mm lens (VS Technology VS-50085/M42), for a total system magnification of 1×.

However, this change in lens was not compatible with the sCMOS camera of the previous wide-area optical system. In a conventional sCMOS camera, there is a microlens array in front of the pixels, to focus incident light onto each pixel. However, this microlens array has a limited optical acceptance angle of ±15°. In a lossless optical system, the product of magnification and NA is a constant. Since the magnification of the new optical system was 1× and the collection NA was 0.5, the light reached the camera with an NA of 0.5, corresponding to an angular range of ±30°, beyond the acceptance angle of conventional sCMOS cameras.

For this reason, a back-illuminated sCMOS camera (Teledyne Kinetix) which did not have an internal microlens array and therefore could accept incident light at high angles was used in the new system.

### Example 3

In this Example, a DMD and a camera were registered in time and space to achieve interleaving of two or more patterns on the DMD while synchronously modulating the wavelength and/or intensity of light impinging on the DMD.

For three-dimensional samples, structured illumination imaging techniques can provide optical sectioning, reduce background fluorescence, and minimize optical heating and photodamage to the sample. These techniques generally require patterning both the light for stimulation and the light for imaging.

For these reasons, it is often desirable to project two or more patterns of illumination at two or more wavelengths concurrently. This goal can be achieved with a digital micromirror device (DMD). To display two concurrent patterns, two separate DMDs can be used, and the images from each can be combined. However, this approach is costly and is limited to two concurrent patterns or wavelengths.

An alternative is to interleave two or more patterns on the same DMD while synchronously modulating the illumination wavelengths. Provided that the response kinetics of the light-sensitive molecular actuators are slower than the cycling frequency of the DMD, the light-responsive molecules effectively experience the time-average illumination. To implement this approach, the images displayed on the DMD must be precisely registered in time and space with the images acquired on the camera. Methods and apparatuses for performing this registration are described below.

### Registration in Time

Many optogenetics and imaging protocols require illumination of a sample with more than one wavelength, intensity, or spatial pattern of light. This goal can be achieved by rapidly switching between DMD patterns while simultaneously modulating the wavelength or intensity of the light impinging on the DMD. However, if a camera is being used to record images of the sample and the transitions in illumination are not synchronized with the camera exposures, then aliasing artifacts can lead to spurious images on the camera and can introduce apparent frame-to-frame variations in the illumination dose.

To address this challenge, a method was developed for precisely synchronizing transitions between multiple illumination patterns and the frame triggers of a high-speed scientific CMOS camera. The core idea was to subdivide each camera frame into multiple epochs, each of which had a different illumination pattern. This approach used the fact that the DMD can be switched faster (typically 10-30 kHz) than the frame rate of the camera (typically 0.5-1 kHz).

Since the DMD takes finite time (about 20 microseconds) to switch between one pattern and the next, all illumination was turned off during the switching time to avoid crosstalk between spatial patterns.

Furthermore, many optogenetic modulators (e.g., actuators, inhibitors) respond on a timescale slower than about 5 ms, so the modulators respond to the time-average illumination over about 5 ms, rather than to the rapid sub-millisecond fluctuations.

Timing modes: In some scientific CMOS cameras (e.g., the Teledyne Kinetix), the camera can accept a digital trigger to start a frame of a pre-defined duration. Provided that the next trigger comes after the previous exposure has completed, the camera can be triggered multiple times to acquire a sequence of images. In this mode, a computer-controlled multi-channel Data Acquisition Card (DAC) provides the trigger pulses for the camera frames, the trigger pulses to step between DMD patterns, signals to modulate the light sources, and any signals required to synchronize other aspects of the experiment (e.g. electrophysiology recordings).

In other scientific cameras (e.g., the Hamamatsu Orca Flash), the camera can be triggered by a single “start” pulse to acquire a pre-defined number of images at a pre-defined frame rate. Since the internal clock on the camera is not phase-locked to the internal clock on the DAC, the camera must act as the master clock on the whole acquisition. To achieve this, the camera is configured to produce a “row-clock” output which provides a digital tick each time a row of pixels is read. Typically this clock is 100 kHz or 200 kHz. For a typical camera exposure of 1 ms, this corresponds to 100 or 200 row-clock ticks per frame. The DAC is configured to use the row clock as its master time-base, so all DMD triggers and light modulation signals are referenced to the camera row-clock.

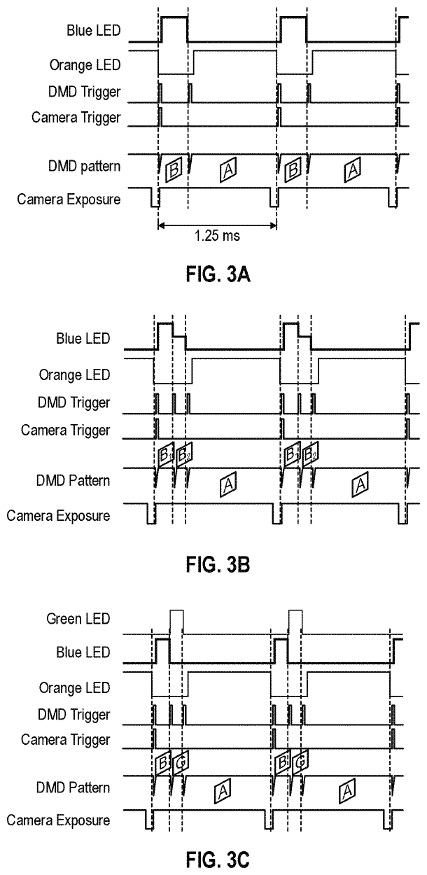

FIGS. 3A-3C illustrate the approach with three examples of methods for synchronizing multiple DMD illumination patterns with a high-speed camera: (1) FIG. 3A shows concurrent illumination with distinct blue and orange patterns; (2) FIG. 3B shows concurrent illumination with two blue patterns at distinct intensities, and a third orange pattern; and (3) FIG. 3C shows concurrent illumination with a blue, green, and orange pattern.

The first method involved near-simultaneous illumination with a blue pattern (B) and an orange pattern (A). A single camera exposure was subdivided into two portions, one for exposure of pattern A and one for exposure of pattern B. To avoid spurious bleedthrough of either illumination wavelength into the other pattern, dark periods of 20-40 microseconds were introduced between the illumination epochs. The DMD was triggered to switch patterns half-way through this dark period. By varying the fraction of time allocated to each pattern, the relative fluxes of light can be adjusted.

This set of patterns may be useful for simultaneous patterned optogenetic stimulation and structured illumination imaging.

The second method involved near-simultaneous illumination with two blue patterns of different intensities (B₁ and B₂) and an orange pattern (A). In this method, the blue exposure epoch was subdivided into two parts. The two parts had different DMD patterns and different drive currents to the LED. This approach permitted illumination of two parts of the sample with different intensities of blue light.

The third method involves near-simultaneous illumination with a blue pattern (B), a green pattern (G), and an orange pattern (A). The DMD cycled among the three patterns, and each LED was only turned on during the interval when the corresponding pattern is presented on the DMD.

Sequencing multiple patterns: The three approaches illustrated in FIGS. 3A-3C can be combined or adjusted to achieve more complex patterns of illumination that are synchronized with the camera. To arrange transitions between illumination regimes (e.g., a period with one blue pattern B₁, followed by a period with a second blue pattern B₂), the DMD can be programmed to alternate between images B₁ and A for a number of camera frames N₁, and then to switch to alternating between images B₂ and A for a number of camera frames N₂. In this way complex sequences of illumination can be composed.

The LEDs can be replaced by lasers, in which case the modulation of the respective wavelengths can be achieved either through direct analog or digital modulation of a laser diode, or via a device such as an acousto-optical tunable filter.

### Registration in Space

Targeted illumination is often intended to illuminate specific biological structures in a sample. These structures can be imaged by a camera, but the pattern of illumination is often specified on the DMD. Therefore, it is desirable to develop a mapping of pixel coordinates on the DMD (or other spatial light modulator) to pixel coordinates on the camera (or other imaging device).

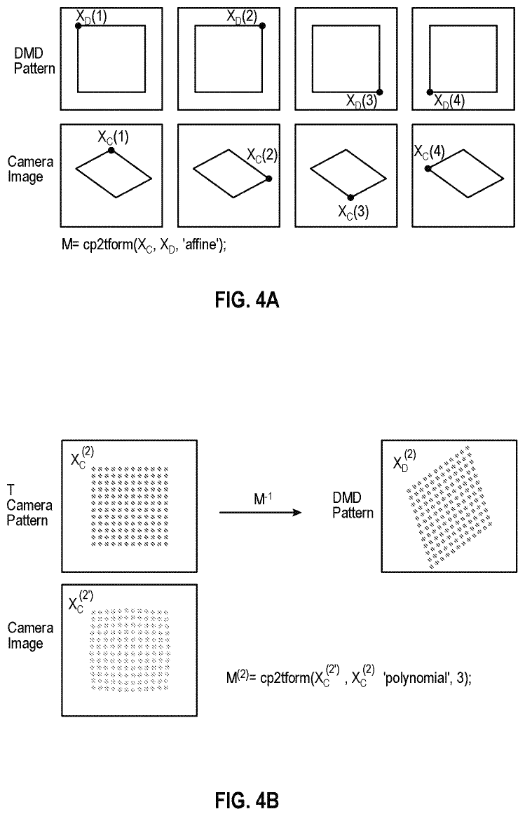

FIGS. 4A-4B show illumination patterns for spatially registering a DMD and a camera. A fluorescent test slide was inserted into the microscope. As shown in FIG. 4A, in the first step, the DMD sequentially illuminated a small group of pixels near each of four vertices of a square, and the camera acquired one image for each illumination pattern. The software then located the centroid of each spot on the camera pixels. These measurements were used to compute a best-fit affine transformation of DMD coordinates (X_{D}) to camera coordinates (X_{C}), for example using the Matlab function cp2tform. The transformation is denoted M(X_{D}→X_{C}). The inverse of this transformation was also calculated, M⁻¹ (X_{C}→X_{D}).

An optional second step was used to increase the accuracy of the mapping of DMD pixels to camera pixels. As shown in FIG. 4B, in the optional second step, a grid of spots,

$${X}_{C}^{( 2 )} ,$$

(e.g., 10×10) was defined in the camera coordinates. These spots were then mapped to DMD coordinates via the transformation M⁻¹ and projected onto the fluorescent test slide. The camera then acquired an image and localized the centroid of each spot to create a grid

$${X}_{C}^{( 2 ′ )} .$$

If the mapping M⁻¹ were perfect, then

$${X}_{C}^{( 2 ′ )} = {X}_{C}^{( 2 )} .$$

However, optical aberrations can lead to differential distortions on the DMD and camera imaging paths, so in general

$${X}_{C}^{( 2 ′ )} \neq {X}_{C}^{( 2 )} .$$

A nonlinear transform, M⁽²⁾, was computed by applying the Matlab functions cp2tform or fitgeotrans to the points

$${X}_{C}^{( 2 ′ )} and {X}_{D}^{( 2 )} .$$

The appropriate order of this transform depends or fitgeotrans to the points on the types of aberrations in the optical system, but typically 3^{rd} or 4^{th} order is sufficient. The nonlinear transform M⁽²⁾ and its inverse were then used for future DMD-to-camera pixel mappings.

### Example 4

In this Example, neuron-selective illumination using registration in time and space between a DMD and a camera was used to map functional connections between neurons in the brain of a mouse (e.g., to determine the influence of an action potential in any neuron i on the membrane potential in each other neuron j).

In a mouse brain expressing a blue-excited channelrhodopsin and an orange-excited voltage indicator protein, specific cells were stimulated in a particular temporal sequence while monitoring the voltage responses of all cells in the circuit. To achieve this, after the DMD and camera were registered in space, the following steps were taken:

1) Structural imaging via structured illumination microscopy (e.g., Hadamard microscopy) was performed using orange light to map the cells expressing the voltage indicator protein.

2) Image segmentation tools were used to identify and locate the coordinates of each expressing neuron.

3) An illumination mask was constructed to target orange light only to the cells of interest. By avoiding illumination of interstitial parts of the tissue, the targeted illumination approach substantially decreased the out-of-focus background fluorescence.

4) A series of illumination masks was constructed to target blue light to cells to be stimulated. By sequentially stimulating subsets of cells and recording the voltage responses of these and other cells, the functional connections between cells was mapped.

5) A stimulation and imaging experiment was run where stimuli and targeted imaging patterns were interleaved with sub-frame time resolution.

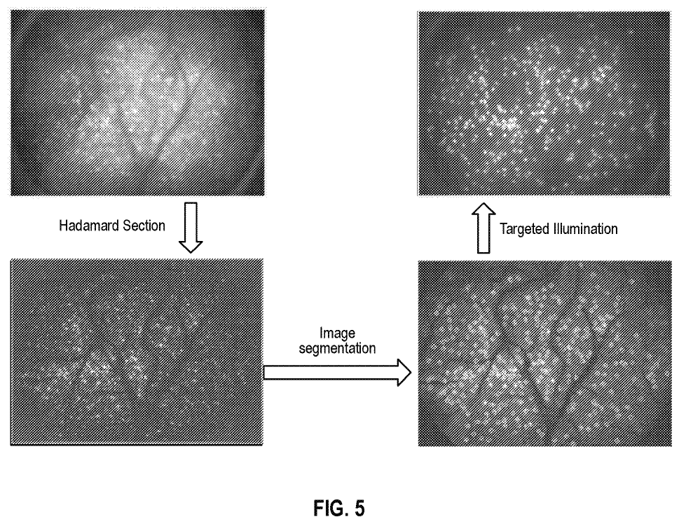

FIG. 5 shows a process for targeted-illumination voltage imaging in the mouse cortex. The images of FIG. 5 show: (A) wide-field epifluorescence; (B) an image of a Hadamard microscopy optical section of the tissue; (C) the locations of identified neurons; and (D) an image of the sample with illumination patterned to only illuminate the identified neurons.

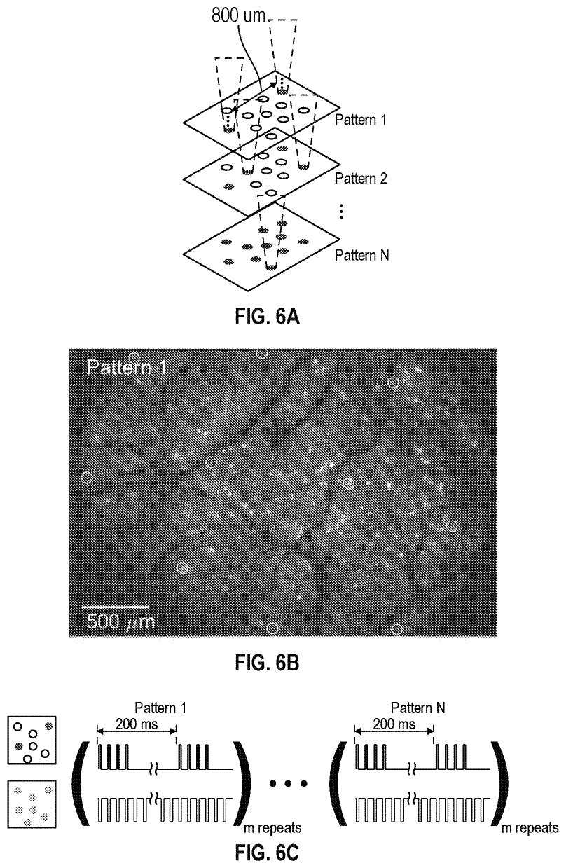

FIG. 6 shows schematic illustrations and images of combining targeted optogenetic stimulation with structured illumination voltage imaging for mapping functional connections. FIG. 6A shows that a series of blue light stimulus patterns were designed to target different subsets of cells. It was recognized that for monosynaptic functional connectivity mapping, simultaneously stimulated cells should be far enough apart (greater than 800 mm in this Example) so that an influence on a putative postsynaptic cell can be assigned with high probability to a specific presynaptic cell. FIG. 6B shows an illumination sequence. During stimulus epochs, blue stimuli (targeted to specific cells) and orange illumination (targeted to all cells) were interleaved. During rest epochs, the blue stimulus was turned off and the orange illumination was maintained. Each stimulus pattern was repeated m times for later construction of a stimulus-triggered average response in each putative postsynaptic cell. The blue stimulus pattern was then changed and the protocol was repeated until all cells had been stimulated. FIG. 6C shows, according to some embodiments, the cells (circled) that were identified for stimulation in Pattern 1.

### Detailed Materials and Methods for Circuit Mapping in Mouse Cortical Layer 1 Microscope

The custom-built wide-area microscope consisted of a pattern projection system and a large field of view (FOV) epifluorescence microscope.

Two LED light sources, blue (Luminus, SBT-70-B) and orange (OSRAM, GY CS8PM1.23) in custom housing were combined via a custom-sized dichroic mirror (Semrock, Di01-R488/543/635, 60×85 mm) and reimaged onto the chip of a digital micromirror device (DMD) module (Vialux V-7001; 1024×768 pixels, 13.7 mm pitch). A total-internal-reflection prism (Young Optics Inc, 72.CAC14G001) was used to direct the light onto the DMD chip. Patterned light was collected by a 100 mm tube lens (Zeiss, Makro-Planar 100 mm), directed into a 50 mm focal length objective lens (Olympus, MVPLAPO 2XC, NA 0.5) via a custom-sized dichroic mirror (Alluxa, 504/605 ULTRA Dualband Dichroic Beamsplitter, 60×85 mm, 5 mm thick substrate), and reimaged onto the sample plane. Fluorescence emission was collected by the objective, filtered by a custom-sized emission filter (Semrock, FF02-641/75, 60 mm diameter), and reimaged onto the camera (Teledyne Photometrics, Kinetix, 3200×3200 pixels) through a 50 mm tube lens (VS Technology, VS-50085/M42). All microscope hardware was synchronized by a data acquisition device (National Instruments, USB-6343) and controlled by custom-written software in MatLab.

### Cranial Window Surgery and Virus Injection

The cranial window implantation procedure follows a protocol previously described in Goldey et al., Removable Cranial Windows for Long-Term Imaging in Awake Mice, NAT. PROTOC., 9:2515-38 (2014). Briefly, a heterozygous NDNF-Cre mouse (P60-120, Jackson Lab, No. 028536) was deeply anesthetized by 3.5% isoflurane, then maintained in anesthesia by 1-1.5% isoflurane. A 3 mm diameter craniotomy was made in the left hemisphere (1.5 mm caudal from bregma, 3.2-3.5 mm from midline). Injections were made with a micropipette (World Precision Instruments, No. 504949) pulled with a pipette puller (Sutter Instruments, P1000). The pipette tip was broken to a diameter of 10-20 μm and beveled with a custom-built pipette beveller. Virus AAV1/9 hSyn-DIO-Voltron-ST-p2a-somCheRiff was diluted to about 3×10¹² GC/ml with PBS. Injections of 60 nL (30-60 nL/min) at a depth of about 150 μm below the dura were performed at 10-14 different injection sites covering the entire exposed cortex. Following the virus injection, a cranial window was placed onto the cortex and glued to the skull with Vetbond (Patterson Veterinary, No. 07-805-5031). The cranial window was made by bonding two layers of 3 mm No. 1 round coverslip (Harvard Apparatus) and one 5 mm No. 1 round coverslip (Harvard Apparatus) with UV curable adhesive (Norland Products, NOA 81). The titanium head plate was bonded to the skull by dental cement (C&B metabond, Parkell, No. 242-3200), and all exposed skull was covered by dental cement. Animals were housed separately to avoid damage to the implant.

### In Vivo Voltage Imaging

### Retroorbital Injection

One day prior to imaging, 100 nanomoles of halo-tag ligand Janelia fluor dye (JFx608) was injected into the retroorbital sinus. To prepare the injection mixture, lyophilized JFx608 was dissolved in 20 μL of dimethyl sulfoxide (DMSO), 20 μL of Pluronic F127 (20% w/v in DMSO), and 60-80 μL of phosphate-buffered saline (PBS).

### Free-Running Treadmill

The treadmill was a modified version of the Janelia design. See Low-Friction Rodent-Driven Belt Treadmill, https://www.janelia.org/open-science/low-friction-rodent-driven-belt-treadmill.

### Head-Fixation and Leveling

To image a large FOV, the cranial window should be level with the sample plane of the microscope objective. To achieve levelness, the head restraint was mounted to a two-axis goniometer (Misumi Inc, B54-60UN, B54-60U2N). Prior to imaging, the head-fixed animal was placed under a custom-built interferometer consisting of a 5 mW 630 nm laser pointer (Amazon), a mirror (Thorlabs, BB1-E02), and a beam splitter (VWR, No. 16004-422). The cranial window acted as the second mirror, and the reflected beams were visualized onto a white target about 4 m away. The two-axis goniometer was adjusted until the two reflected beam spots overlapped on the white target.

### Targeted Illumination

In cortical Layer 1, neurons are sparse in space. Sparse illumination targeted at neuronal somas can drastically reduce the background fluorescence, enhancing signal-to-noise ratio. A semi-automatic pipeline was developed to acquire a high-contrast digital confocal image, segment the image, and generate targeted illumination pattern on the DMD. All calculations were performed in MatLab.

### Hadamard Microscopy

Digital confocal images were acquired via Hadamard microscopy as previously described. See Farhi et al., Wide-Area All-Optical Neurophysiology in Acute Brain Slices. J. NEUROSCI. 39:4889-4908 (2019). In brief, the confocal illumination patterns were generated with the last 63 columns of a size 64 normalized Hadamard matrix and a 13-pixel spatial offset. A calibration dataset was acquired on a thin fluorescent sample under orange illumination. The sample consisted of a layer of pink neon Sharpie (Newell Brands) sandwiched between a three-layer #1 coverslip (VWR, No. 48393-059) bonded by UV curable adhesive (matching the in vivo condition) and a 1 mm glass slide (VWR, No. 16004-422). Then the same patterns were projected and recorded on the live sample with ten repeats. The repeats were then reconstructed and averaged, yielding the final confocal image.

### Image Segmentation and Pattern Generation

The confocal image was filtered by a 7×7 pixel mean-subtracted Gaussian filter with standard deviation of 7 pixel, then segmented using the watershed algorithm. Watershed regions fewer than 10 pixels were discarded. The putative neuron in each watershed region was located using the maximum intensity pixel. 320 neuron-regions most correlated with the Gaussian filter were chosen, and a 40 micrometer wide disk-shaped illumination mask was generated for each neuron. Correlation was calculated using the unfiltered confocal image.

### Functional Connectivity Mapping

The optogenetic stimulation protocol was designed to probe GABAergic and gap junctional connections. For all imaging experiments, targeted neurons were continuously imaged at 787 Hz under orange illumination at about 16 mW/mm². The animal was awake and allowed to run on the treadmill during the imaging session.

### GABAergic Connection

Given previous reports on anatomy and connectivity among L1 interneurons, it was thought that synaptic interactions between interneurons greater than 400 micrometers apart would be negligible. For each 200 ms stimulation period, a set of neurons with mutual spatial separation greater than 800 micrometers were randomly chosen and simultaneously stimulated for 20 ms by about 8 mW/mm² patterned blue stimulation (20 μm wide disks). The stimulation was repeated 14400 times. A rest period of 10 s followed every 30 trials. Each neuron was stimulated about 240 times on average.

### Gap Junctional Connection

Gap junction connections cause the membrane potential of a downstream neuron to closely follow that of a spiking neuron as a delayed and attenuated copy, commonly named spikelet. The amplitude of a spikelet is a few millivolts, and on a single-trial basis it is often difficult to detect in voltage imaging due to shot noise. To facilitate trial-averaging and to decouple gap junctional effect from common excitatory inputs, in each trial, half of all targeted neurons (i.e., 160 putative neurons) were optogenetically stimulated with patterned (20 μm wide disks), 2-4 mW/mm², 750 ms long blue step pulse. The choice of stimulated neurons was drawn from one column of a Hadamard matrix of size 320. Stimulation was repeated 1280 times with the Hadamard matrix repeated 4 times.

The stimulation trials for the two types of connectivity were ordered as follows: 90 trials of GABAergic connection mapping followed by 8 trials of gap junctional connection mapping.

### Mechanical Drift Correction

The connectivity mapping experiment lasted 2:30 h. Without intervention, neurons can slowly move outside of the illumination mask due to mechanical drift. To correct for this drift, a Hadamard image was taken every 6 minutes. Drifts in lateral directions were corrected using displacement vectors calculated by the Farneback optical flow algorithm. Drifts in the vertical direction were corrected using displacements calculated by correlating the fast Fourier transform of the confocal image with that of a 60 mm deep confocal z-stack (3 mm resolution). The most correlated depth was found by fitting the correlation with a quadratic polynomial. The z-stack was taken prior to the connection mapping trial.

### Temporal Multiplexing for Independent Dual Color Illumination

Since blue stimulation and orange illumination patterns were generated by the same DMD, dual color illumination with distinct patterns required orthogonal timing for the two colors. To avoid flicker artifacts in the recording, each DMD pattern onset was synchronized with the light pulse onset, and the light pulse onset was synchronized with the camera exposure onset. For each camera exposure, 80% was allocated to the orange pattern/light pair, and 20% was allocated to the blue pattern/light pair.

### Data Analysis

All analysis was performed in MatLab.

Extracting Fluorescence Time Trace from Voltage Recording

Fluorescence movie recordings contain both voltage-sensitive fluorescence signals and motion-related spatial displacements. Spatial displacements were corrected by applying the Farneback optical flow algorithm on temporally smoothed movies. Savitzky-Golay filter of 2^{nd} order over a 9-frame temporal window was used for temporal smoothing.

The signal extraction problem for voltage imaging has been approached in several ways in recent years, but not for the imaging regime of this Example (i.e., ultra-low magnification with targeted illumination). Here, the recent approaches were combined along with modifications to suit the experimental conditions.

The general approach falls under the framework of non-negative matrix factorization (NMF), where the fluorescence movie is modeled as

$$y = A {C}^{T} + b {f}^{T}$$

where Y is the vectorized movie (d×T matrix), A=[a₁ a₂ . . . aₙ], aᵢ∈ [symbol] is the spatial footprint of neurons, C=[c₁ c₂ . . . cₙ], cᵢ∈ [symbol] is the fluorescence signal of neurons, b∈ [symbol] is the spatial footprint of the background fluorescence (i.e., out of focus fluorescence of nearby neurons, neuronal processes, etc.), and f∈ [symbol] is the fluorescence signal of the background. An extra term, DM^{T}, was added to capture fluorescence fluctuations from a point emitter moving under targeted illumination patterns, i.e., motion artifacts:

$$y = A {C}^{T} + b {f}^{T} + D {M}^{T}$$

Where D=[d₁ d₂ . . . dₗ], dᵢ∈ [symbol] is the spatial footprint of the motion artifacts and M=[m₁ m₂ . . . mₗ], mᵢ ∈ [symbol] is the motion artifact signal.

To solve the matrix factorization problem for hundreds of neurons, the spatial compactness of the voltage sources (i.e., neurons with a soma-targeted voltage indicator) was leveraged, and the approach of constrained NMF, where NMF was solved only for a small 25×25 pixel field of view (FOV) over each neuron, was followed.

Given that good initializations are important for NMF, the initialization schemes for all NMF components are described.

### Initialization of Parameters A, C:

Spatial/temporal matrices for neurons were initialized via two steps: local principal component analysis (PCA) and spike time regression. Taking advantage of the digital confocal image, the image segmentation results (from the imaging step) were used to do local PCA on segmented ROI (5-pixel wide disk):

$${Y}_{ROI} = U \sum {V}^{T}$$

where the first component of U, u₁, was the spatial footprint of a neuron, although u₁ was contaminated by background, motion artifacts, and overlapping neuronal signals. Nevertheless, the time trace

$$t = {u}_{1}^{T} Y$$

contained spike signals. Inspired by the effectiveness of spike time regression to obtain spatial footprints, the top 500 spikes in t were used to construct the spike time trace:

$${t}_{spike} ( t ) = \{ \begin{matrix}1 , & where there is a spike at t \\ 0 , & otherwise\end{matrix}$$

and regression was performed to obtain the refined footprint:

$${u}_{r} = {( {t}_{spike}^{T} {t}_{spike} )}^{- 1} {Yt}_{spike}$$

Repeating the above process again, u_{final} was obtained.

At this stage, u_{final} was free of background and motion artifact contamination, but not overlapping neurons. Furthermore, even without spatial overlap, gap junction connections can still lead to nearby neuron contamination in u_{final}. Therefore, as a final step, watershed segmentation was performed on u_{final} to obtain A₀, and tₛₚᵢₖₑ was chosen to be C₀.

### Initialization of Parameters b, f:

It was assumed that: (1) background fluorescence was spatially homogeneous across the 25×25 pixel (160×160 micrometer²) FOV, and (2) the spatial footprint of a neuron was no larger than a 60 micrometer diameter disk. To obtain background signal, f₀, the pixels 30 micrometers away from neurons were averaged. The background footprint was then obtained as regression weights

$${b}_{0} = {( {f}_{0}^{T} {f}_{0} )}^{- 1} {Yf}_{0}$$

### Initialization of Parameters D, M:

A moving emitter under a static illumination spot produces a motion-correlated fluorescence time trace. In general, perfect overlap between neurons and the DMD illumination patterns is not guaranteed, and the magnitude of motion-correlation varies among neurons. Furthermore, tissue is inherently non-rigid, and the magnitude/phase of displacements can vary among neurons. To account for these variabilities, it was sought to produce a set of basis vectors for motion-related artifacts. Displacement vectors x(t), y(t) were first collected for all cells by averaging the velocity vector field over a small ROI around each cell (6-pixel wide disk). Since optical flow is an intensity-based algorithm, shot-noise was present in displacement vectors. A Gaussian low pass filter (60 Hz cutoff) was applied to all displacement vectors. Given that motion-related fluorescence variation is non-linearly related to the displacement, such non-linearity was approximated with a quadratic model:

$${F}_{m} = {a}_{1} x + {a}_{2} y + {a}_{3} x y + {a}_{4} {x}^{2} + {a}_{5} {y}^{2}$$

To generate an orthogonal basis set, singular value decomposition was computed over

$${[ {q}_{1} {q}_{2} … {q}_{n} ]}^{T} = U \sum {V}^{T}$$

$$where$$

$${q}_{i} = [ {x}_{i} {y}_{i} {x}_{i} {y}_{i} {x}_{i}^{2} {y}_{i}^{2} ] , for cell i$$

where U is the basis set. However, U has large dimensions, and the covariance decays rapidly over the first tens of components. For each cell, 20 components, u, most correlated (i.e., dot product) to the raw fluorescence of the cell were selected, where the raw fluorescence was produced by averaging over a 6-pixel wide disk around the cell. Motion has distinct frequency signatures depending on its sources (e.g., heartbeat, breathing, locomotion, etc.). The 20 components were further separated into high and low frequency components:

$${M}_{0} = [ {u}_{L} {u}_{H} ]$$

$${u}_{H} = u - {u}_{L} ,$$

where u_{L} is low-pass filtered u with a 30-Hz cut-off Gaussian filter. The motion artifact footprint was obtained as regression weights:

$${D}_{0} = {( {M}_{0}^{T} {M}_{0} )}^{- 1} {YM}_{0}$$

### Solving NMF

For each cell, the matrix decomposition problem was solved on a 25×25 pixel patch using the HALS algorithm with modified constraints:

$${min}_{A \geq 0 , C , b \geq 0 , f , d} \frac{1}{2} {\vert Y - AC - bf - DM \vert}^{2}$$

Due to gap junction connection among interneurons, additional spatial constraints were introduced on A=[a₁ a₂ . . . aₙ], where aᵢ≥0 for pixels within a 60 mm disk, and

$${a}_{i}^{T} {a}_{j} = 0 , if i \neq j .$$

### Example 5

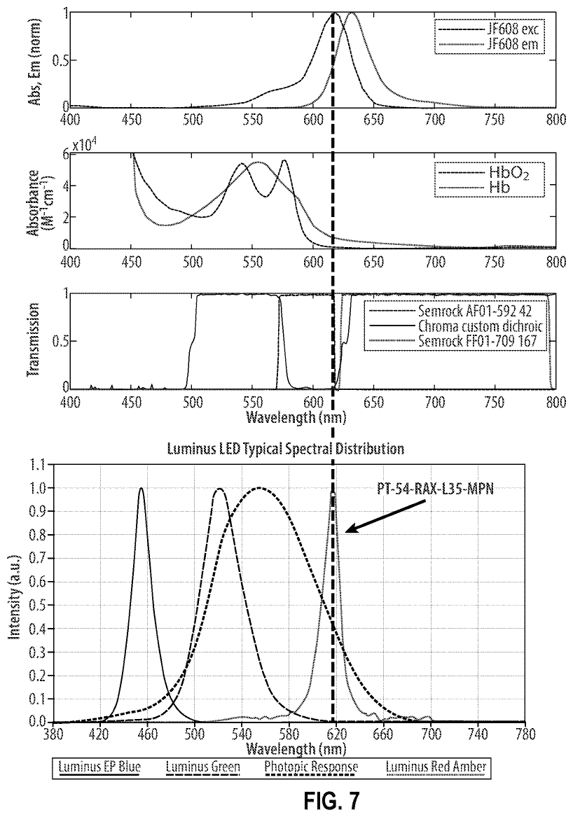

An example spectral design is provided herein. FIG. 7 shows an exemplary design of filter spectra for a wide-area in vivo voltage imaging system. In the illustrated embodiment, the design is optimized to avoid light absorption by hemoglobin (Hb) or oxygenated hemoglobin (HbO2), while efficiently exciting the dye JF608 which is a highly efficient and bioavailable tag for the Voltron and Voltron2 genetically encoded voltage indicators.

The plotted hemoglobin spectra show that the absorption of both oxygenated and deoxygenated hemoglobin drops sharply for wavelengths>600 nm. The light source is an LED (Luminus PT-54-RAX-L35-MPN) whose emission spectrum closely matches the JF608 absorption spectrum (the other plots on the LED emission spectrum plot are not relevant here). The excitation filter is designed to remove the blue edge of the LED emission which might otherwise spuriously activate a channelrhodopsin co-expressed with the voltage indicator. The excitation filter also removes the red edge of the LED emission which would otherwise pass through the emission filter and contaminate the fluorescence signal at the camera. Accordingly, in some embodiments, a light source module comprises an excitation filter that filters at least some blue wavelengths and at least some red wavelengths in the beam of light emitted by the light source.

The dichroic mirror reflects light to the sample at wavelengths 575-620 nm. The dichroic mirror also reflects at wavelengths<500 nm, which could be used for blue-light excitation or for violet light-mediated photochemistry. The emission filter transmits fluorescence from the sample at wavelengths 625-792 nm.

This design of the illustrated embodiment is optimized to minimize loss of light (either fluorescence excitation or emission) by hemoglobin absorption, while maximizing voltage-sensitive signal through efficient excitation of the voltage indicator.

### Example 6

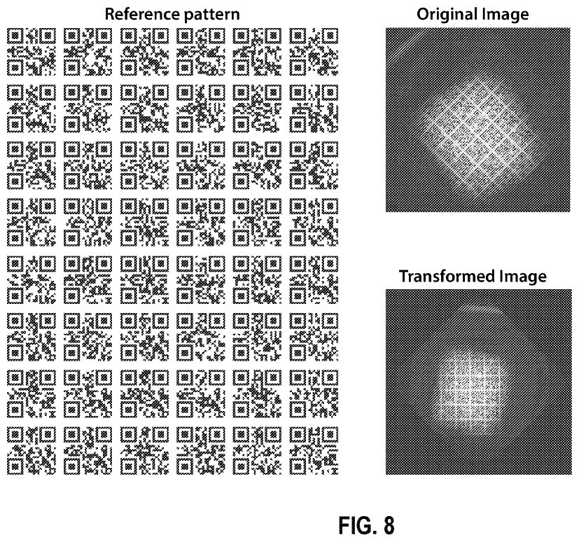

An example for spectral registration of a light-modulator and camera is provided herein. FIG. 8 shows a workflow for registering a spatial light-patterning device to a camera. For example, the spatial light-patterning device may be a digital micromirror device (DMD) or a liquid crystal spatial light modulator. In the illustrated example, a homogeneous fluorescent exposure target is loaded into the microscope. The light-patterning device projects a grid of quick-response (QR) codes onto the target. The pattern encodes the digits 1-N, where N is the number of QR codes. The camera acquires an image of the projected pattern. Software reads all of the visible QR codes, and localized the fiducial marks in each. The pattern of fiducial marks is then used to calculate a transformation from the pixel coordinates of the light-patterning device to the pixel coordinates of the camera. A transformed pattern of QR codes is projected onto the exposure target to confirm the accuracy of the mapping.

Accordingly, some embodiments of the technology described herein provides for a method of registering a light-patterning device to an image capturing device is provided. In some embodiments. The method may comprise loading a sample target onto an imaging device; projecting, with the light-patterning device, a pattern of quick response (QR) codes onto the sample target, the pattern of QR codes encoding a plurality of digits corresponding to a number of QR codes in the pattern; acquiring, with the image capturing device, an image of the pattern of QR codes projected onto the sample target; determining a pattern of fiducial marks in the pattern of QR codes based on the acquired image of the pattern of QR codes; and calculating, using the determined pattern of fiducial marks in the pattern of QR codes, a transformation from pixel coordinates of the light-patterning device to pixel coordinates of the image capturing device to obtain a transformed pattern of QR codes. In some embodiments, the method further comprises projecting the transformed pattern of QR codes onto the exposure target and confirming accuracy of the transformed pattern of QR codes based on the projecting the transformed pattern. In some embodiments, the image capturing device comprises a camera. In some embodiments, the imaging device comprises a microscope. In some embodiments, the light-patterning device comprises a digital micromirror device. In some embodiments, the light-patterning device comprises a liquid crystal spatial light modulator.

While several embodiments have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. For example, while embodiments based on an epifluorescence microscope were described in detail, Hadamard microscopy may be used with other types of microscopes. For example, some embodiments may use similar techniques with transmitted and/or scattered light microscopes, such as white light microscopes. In such embodiments, optical sections are created using light that is transmitted through a sample rather than light that is fluorescently emitted by the sample.

More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A light source module, comprising:
a light source configured to emit a beam of light;
an enclosure, wherein the light source is positioned within the enclosure, wherein a surface of the enclosure comprises an optical window positioned within a path of the beam of light, and wherein an interior volume of the enclosure comprises an inert gas;
a thermoelectric cooler in thermal communication with the enclosure; and
a heat sink in thermal communication with the thermoelectric cooler.

2. The light source module according to claim 1, wherein the light source comprises an LED.

3. The light source module according to any one of claims 1-2, wherein the light source comprises a single LED.

4. The light source module according to any one of claims 1-3, wherein the light source is configured to be driven at a current of at least 1 A during operation of the light source module.

5. The light source module according to any one of claims 1-4, wherein the light source is configured to be operated at a temperature of −30° C. or less during operation of the light source module.

6. The light source module according to any one of claims 1-5, wherein the light source is configured to emit the beam of light at an intensity of at least 2 W/cm² at the sample plane during operation of the light source module.

7. The light source module according to any one of claims 1-6, further comprising a first collimating lens positioned between the light source and the optical window of the enclosure.

8. The light source module according to any one of claims 1-7, further comprising an excitation filter positioned between the light source and the optical window of the enclosure.

9. The light source module according to claim 8, wherein the excitation filter filters at least some blue wavelengths and at least some red wavelengths in the beam of light emitted by the light source.

10. The light source module according to any one of claims 1-9, further comprising a plano-convex lens positioned outside the enclosure and within the path of the beam of light.

11. The light source module according to any one of claims 1-10, wherein the heat sink is a water-cooled heat sink.

12. The light source module according to any one of claims 1-11, wherein the thermoelectric cooler is in direct physical contact with the enclosure.

13. The light source module according to any one of claims 1-12, wherein the thermoelectric cooler is in direct physical contact with the heat sink.

14. An imaging system, comprising:
a first light source module configured to emit a first beam of light at a first wavelength;
a second light source module configured to emit a second beam of light at a second wavelength;
a first optical system configured to project at least a portion of the first beam of light and/or the second beam of light onto a spatial light modulator configured to produce a spatial pattern of illumination;
a second optical system configured to project the spatial pattern of illumination onto a sample being imaged;
a back-illuminated CMOS camera having an optical acceptance angle of at least 16°, positioned to receive light from the sample being imaged; and
a controller configured to modulate current through the first light source module and/or the second light source module, control the spatial light modulator to project a sequence of spatial patterns of illumination onto the sample over time, and/or control exposures of the camera.

15. The imaging system of claim 14, wherein the first wavelength is different from the second wavelength.

16. The imaging system of any one of claims 14-15, wherein the first optical system comprises a dichroic beam combiner.

17. The imaging system of any one of claims 14-16, wherein the first optical system comprises a total internal reflection prism.

18. The imaging system of any one of claims 14-17, wherein the first optical system comprises a plano-convex lens.

19. The imaging system of any one of claims 14-18, wherein the spatial light modulator comprises a digital micromirror device.

20. The imaging system of claim 19, wherein the digital micromirror device comprises at least 786,432 micromirrors.

21. The imaging system of any one of claims 14-20, wherein the second optical system comprises a projection lens.

22. The imaging system of any one of claims 14-21, wherein the second optical system comprises a dichroic mirror.

23. The imaging system of any one of claims 14-22, wherein the second optical system comprises an objective lens.

24. The imaging system of any one of claims 14-23, further comprising an emission filter positioned within an optical path between the back-illuminated CMOS camera and the sample being imaged.

25. The imaging system of any one of claims 14-24, further comprising a 50 mm tube lens positioned within an optical path between the back-illuminated CMOS camera and the sample being imaged.

26. The imaging system according to any one of claims 14-25, wherein the back-illuminated CMOS camera has an optical acceptance angle of at least 20°.

27. The imaging system according to any one of claims 14-26, wherein the back-illuminated CMOS camera has an optical acceptance angle of at least 30°.

28. The imaging system of any one of claims 14-27, wherein the back-illuminated CMOS camera does not comprise an internal microlens array.

29. The imaging system according to any one of claims 14-28, wherein the back-illuminated CMOS camera has a frame rate of at least 400 Hz during operation of the imaging system.

30. The imaging system of any one of claims 14-29, wherein a magnification of the imaging system is in a range from 1× to 1.5×.

31. The imaging system according to any one of claims 14-30, wherein the first light source module and/or the second light source module are configured to emit light having an intensity of at least 2 W/cm² at the sample plane.

32. The imaging system according to any one of claims 14-31, wherein the imaging system is configured to image an area having a largest dimension of at least 2 mm.

33. A method of projecting two or more spatial patterns of illumination at two or more wavelengths concurrently, comprising:
initiating an exposure of an imaging device for an exposure interval, wherein the exposure interval comprises a plurality of sub-intervals;
controlling a first light source to emit light at a first wavelength during a first sub-interval of the exposure interval;
controlling a spatial light modulator to project a first spatial pattern of light from the first light source onto a sample during the first sub-interval;
controlling a second light source to emit light at a second wavelength during a second sub-interval of the exposure interval;
controlling the spatial light modulator to project a second spatial pattern of light from the second light source onto the sample during the second sub-interval; and
controlling the first light source and the second light source such that the sample is not illuminated during a third sub-interval between the first sub-interval and the second sub-interval.

34. The method of claim 33, wherein the spatial light modulator comprises a digital micromirror device.

35. The method of any one of claims 33-34, wherein the first wavelength is in a range of 450 nm to 495 nm.

36. The method of any one of claims 33-34, wherein the first sub-interval has a duration that is 10% to 30% of a duration of the exposure interval.

37. The method of any one of claims 33-36, wherein the second wavelength is in a range of 590 nm to 625 nm.

38. The method of any one of claims 33-36, wherein the second wavelength is in a range from 625 nm to 750 nm.

39. The method of any one of claims 33-38, wherein the second sub-interval has a duration that is 60% to 90% of a duration of the exposure interval.

40. The method of any one of claims 33-39, wherein the exposure interval has a duration in a range from 1 ms to 5 ms.

41. The method of any one of claims 33-40, wherein the first spatial pattern comprises a plurality of sub-patterns and the first sub-interval comprises a plurality of sub-sub-intervals, wherein the method further comprises controlling the spatial light modulator to project a first sub-pattern at the first wavelength during a first sub-sub-interval of the first sub-interval and controlling the spatial light modulator to project a second sub-pattern at the first wavelength during a second sub-sub-interval of the first sub-interval.

42. The method of any one of claims 33-41, further comprising controlling a third light source to emit light at a third wavelength during a fourth sub-interval of the exposure interval and controlling the spatial light modulator to project a third spatial pattern of light at a third wavelength onto the sample during the fourth sub-interval.

43. The method of claim 42, wherein the third wavelength is in a range from 495 nm to 565 nm.

44. The method of any one of claims 42-43, wherein the fourth sub-interval has a duration that is 10% to 30% of a duration of the exposure interval.

45. A method of imaging a sample, comprising:
illuminating a biological sample with a first sequence of spatial patterns using light of a first wavelength, wherein the biological sample comprises a plurality of cells expressing one or more fluorescent reporters and one or more light-activated molecules;
capturing image data based on light of a second wavelength emitted by the sample using an imaging device;
constructing a second spatial pattern configured to provide targeted illumination of cells of the plurality of cells expressing the one or more fluorescent reporters;
constructing a third sequence of spatial patterns, wherein each spatial pattern of the third sequence of spatial patterns is configured to provide targeted illumination of a subset of cells expressing the one or more fluorescent reporters; and
illuminating the biological sample with the second spatial pattern at the first wavelength and one or more spatial patterns of the third sequence of spatial patterns at a third wavelength during a stimulus period, wherein illumination with the second spatial patterns alternates with illumination with a spatial pattern of the third sequence of spatial patterns at a rate higher than a frame rate of the imaging device.

46. The method of claim 45, wherein the cells are neurons or myocytes.

47. The method of any one of claims 45-46, wherein the one or more fluorescent reporters comprise one or more voltage reporters, calcium reporters, and/or cAMP reporters.

48. The method of any one of claims 45-47, wherein the one or more light-activated molecules comprise a channelrhodopsin and/or an adenylyl cyclase.

49. The method of any one of claims 45-48, wherein the first sequence of spatial patterns is generated using a Hadamard matrix.

50. A method for registering a light-patterning device to an image capturing device, the method comprising:
loading a sample target onto an imaging device;
projecting, with the light-patterning device, a pattern of quick response (QR) codes onto the sample target, the pattern of QR codes encoding a plurality of digits corresponding to a number of QR codes in the pattern;
acquiring, with the image capturing device, an image of the pattern of QR codes projected onto the sample target;
determining a pattern of fiducial marks in the pattern of QR codes based on the acquired image of the pattern of QR codes;
calculating, using the determined pattern of fiducial marks in the pattern of QR codes, a transformation from pixel coordinates of the light-patterning device to pixel coordinates of the image capturing device to obtain a transformed pattern of QR codes.

51. The method of claim 50, further comprising projecting the transformed pattern of QR codes onto the exposure target and confirming accuracy of the transformed pattern of QR codes based on the projecting the transformed pattern.

52. The method of any one of claims 50-51, wherein the image capturing device comprises a camera.

53. The method of any one of claims 50-52, wherein the imaging device comprises a microscope.

54. The method of any one of claims 50-53, wherein the image capturing device comprises a camera.

55. The method of any one of claims 50-54, wherein the imaging device comprises a microscope.
